(19) United States
(12) Patent Application Publication
(10) Pub. No.: US 2002/0160459 A1
(43) Pub. Date: Oct. 31, 2002

(54) PROCESS FOR PRODUCTION OF N-GLUCOSAMINE

(76) Inventors: ALAN BERRY, BLOOMFIELD, NJ (US); RICHARD P. BURLINGAME, MANITOWOC, WI (US); JAMES R. MILLIS, KOHLER, WI (US)

Correspondence Address: SHERIDAN ROSS PC, 1560 BROADWAY, SUITE 1200, DENVER, CO, 80202

(21) Appl. No.: 09/341,600

(22) PCT Filed: Jan. 14, 1998
(86) PCT No.: PCT/US98/00800

(51) Int. Cl. C12P 1/00

(52) U.S. Cl. 435/72; 435/243

(57) ABSTRACT

The present invention relates to a method for producing N-glucosamine by fermentation of a genetically modified microorganism.

### FIELD OF THE INVENTION

The present invention relates to a method for producing N-glucosamine by fermentation. The present invention also relates to genetically modified strains of microorganisms useful for producing N-glucosamine.

### BACKGROUND OF THE INVENTION

Amino sugars are usually found as monomer residues in complex oligosaccharides and polysaccharides. N-glucosamine is an amino derivative of the simple sugar, glucose. N-glucosamine and other amino sugars are important constituents of many natural polysaccharides. For example, polysaccharides containing amino sugars can form structural materials for cells, analogous to structural proteins.

N-glucosamine is manufactured as a nutraceutical product with applications in the treatment of osteoarthritic conditions in animals and humans. The market for N-glucosamine is experiencing tremendous growth. Furthermore, significant erosion of the world market price for N-glucosamine is not expected.

N-glucosamine is currently obtained by acid hydrolysis of chitin, a complex carbohydrate derived from N-acetyl-D-glucosamine. Alternatively, N-glucosamine can also be produced by acid hydrolysis of variously acetylated chitosans. These processes suffer from poor product yields (in the range of 50% conversion of substrate to N-glucosamine). Moreover, the availability of raw material (i.e., a source of chitin, such as crab shells) is becoming increasingly limited.

Therefore, there is a need in the industry for a cost-effective method for producing high yields of N-glucosamine for commercial sale and use.

### SUMMARY OF THE INVENTION

One embodiment of the present invention relates to a method to produce N-glucosamine by fermentation of a microorganism. This method includes the steps of: (a) culturing in a fermentation medium a microorganism having a genetic modification in an amino sugar metabolic pathway; and (b) recovering a product produced from the step of culturing which is selected from the group of N-glucosamine-6-phosphate and N-glucosamine. Such an amino sugar metabolic pathway is selected from the group of a pathway for converting N-glucosamine-6-phosphate into another compound, a pathway for synthesizing N-glucosamine-6-phosphate, a pathway for transport of N-glucosamine or N-glucosamine-6-phosphate out of the microorganism, a pathway for transport of N-glucosamine into the microorganism, and a pathway which competes for substrates involved in the production of N-glucosamine-6-phosphate. The fermentation medium includes assimilable sources of carbon, nitrogen and phosphate. In a preferred embodiment, the microorganism is a bacterium or a yeast, and more preferably, a bacterium of the genus Escherichia, and even more preferably, Escherichia coli.

In one embodiment, the product can be recovered by recovering intracellular N-glucosamine-6-phosphate from the microorganism and/or recovering extracellular N-glucosamine from the fermentation medium. In further embodiments, the step of recovering can include purifying N-glucosamine from the fermentation medium, isolating N-glucosamine-6-phosphate from the microorganism, and/or dephosphorylating the N-glucosamine-6-phosphate to produce N-glucosamine.

In yet another embodiment, the step of culturing includes the step of maintaining the carbon source at a concentration of from about 0.5% to about 5% in the fermentation medium.

In a preferred embodiment, the microorganism has a modification in a gene which encodes a protein including, but not limited to, N-acetylglucosamine-6-phosphate deacetylase, N-glucosamine-6-phosphate deaminase, N-acetyl-glucosamine-specific enzyme II^{Nag}, N-glucosamine-6-phosphate synthase, phosphoglucosamine mutase, N-glucosamine-1-phosphate acetyltransferase-N-acetylglucosamine-1-phosphate uridyltransferase, phosphofructokinase, enzyme II^{Glc} of the PEP:glucose PTS, EIIM, P/III^{Man} of the PEP:mannose PTS, or alkaline phosphatase.

In another embodiment, the genetic modification includes the transformation of the microorganism with a recombinant nucleic acid molecule encoding N-glucosamine-6-phosphate synthase to increase expression of the N-glucosamine-6-phosphate synthase by the microorganism. The recombinant nucleic acid molecule is operatively linked to a transcription control sequence. In a further embodiment, the recombinant nucleic acid molecule is integrated into the genome of the microorganism. In yet another embodiment, the recombinant nucleic acid molecule encoding N-glucosamine-6-phosphate synthase has a genetic modification which reduces N-glucosamine-6-phosphate product inhibition of the N-glucosamine-6-phosphate synthase. In another embodiment, such a microorganism has an additional genetic modification in genes encoding N-acetylglucosamine-6-phosphate deacetylase, N-glucosamine-6-phosphate deaminase and N-acetyl-glucosamine-specific enzyme II^{Nag}, wherein the genetic modification decreases enzymatic activity of the protein.

Another embodiment of the present invention relates to a method to produce N-glucosamine by fermentation which includes the steps of (a) culturing an Escherichia coli transformed with a recombinant nucleic acid molecule encoding N-glucosamine-6-phosphate synthase in a fermentation medium comprising assimilable sources of carbon, nitrogen and phosphate to produce a product, and (b) recovering the product. The product includes intracellular N-glucosamine-6-phosphate which is recovered from the Escherichia coli and/or extracellular N-glucosamine which is recovered from the fermentation medium. In this embodiment, the recombinant nucleic acid molecule increases expression of the N-glucosamine-6-phosphate synthase by the Escherichia coli, and is operatively linked to a transcription control sequence. In one embodiment, the recombinant nucleic acid molecule comprises a genetic modification which reduces N-glucosamine-6-phosphate product inhibition of the N-glucosamine-6-phosphate synthase. In another embodiment, the Escherichia coli has an additional genetic modification in at least one gene selected from the group of nagA, nagB, nagC, nagD, nagE, manXYZ, glmM, pfkB, pfkA, glmU, glmS, ptsG and/or alkaline phosphatase gene.

Yet another embodiment of the present invention relates to a microorganism for producing N-glucosamine by a biosynthetic process. The microorganism is transformed with a recombinant nucleic acid molecule encoding N-glucosamine-6-phosphate synthase, wherein the recombinant nucleic acid molecule is operatively linked to a transcription control sequence. The recombinant nucleic acid molecule further comprises a genetic modification which reduces N-glucosamine-6-phosphate product inhibition of the N-glucosamine-6-phosphate synthase. The expression of the recombinant nucleic acid molecule increases expression of the N-glucosamine-6-phosphate synthase by the microorganism. In a preferred embodiment, the recombinant nucleic acid molecule is integrated into the genome of the microorganism. In yet another embodiment, the microorganism has at least one additional genetic modification in a gene encoding a protein selected from the group consisting of N-acetylglucosamine-6-phosphate deacetylase, N-glucosamine-6-phosphate deaminase, N-acetyl-glucosamine-specific enzyme II^{Nag}, phosphoglucosamine mutase, N-glucosamine-1-phosphate acetyltransferase-N-acetylglucosamine-1-phosphate uridyltransferase, phosphofructokinase, Enzyme II^{Glc} of the PEP:glucose PTS, EIIM, P/III^{Man} of the PEP:mannose PTS, and/or alkaline phosphatase, wherein the genetic modification decreases enzymatic activity of the protein. In yet another embodiment, the microorganism has a modification in genes encoding N-acetylglucosamine-6-phosphate deacetylase, N-glucosamine-6-phosphate deaminase and N-acetyl-glucosamine-specific enzyme II^{Nag}, wherein the genetic modification decreases enzymatic activity of the protein. In a preferred embodiment, the genetic modification is a deletion of at least a portion of the genes.

In a further embodiment, the microorganism is Escherichia coli, having a modification in a gene selected from the group of nagA, nagB, nagC, nagD, nagE, manXYZ, glmM, pfkB, pfkA, glmU, ptsG and/or alkaline phosphatase gene. In one embodiment, such an Escherichia coli has a deletion of nag regulon genes, and in another embodiment, such an Escherichia coli has a deletion of nag regulon genes and a genetic modification in manXYZ genes such that the proteins encoded by the manXYZ genes have decreased enzymatic activity.

Yet another embodiment of the present invention is a microorganism as described above which produces at least about 20 mg/L of N-glucosamine when cultured for about 24 hours at 37° C. to a cell density of at least about 8 g/L by dry cell weight, in a pH 7.0 fermentation medium comprising: 14 g/L K₂HPO₄, 16 g/L KH₂PO₄, 1 g/L Na₃Citrate.2H₂O, 5 g/L (NH₄)₂SO₄, 20 g/L glucose, 10 mM MgSO₄, 1 mM CaCl₂, and 1 mM IPTG.

Another embodiment of the present invention is a microorganism for producing N-glucosamine by a biosynthetic process, which includes: (a) a recombinant nucleic acid molecule encoding N-glucosamine-6-phosphate synthase operatively linked to a transcription control sequence; and, (b) at least one genetic modification in a gene encoding a protein selected from the group of N-acetylglucosamine-6-phosphate deacetylase, N-glucosamine-6-phosphate deaminase, N-acetyl-glucosamine-specific enzyme II^{Nag}, phosphoglucosamine mutase, N-glucosamine-1-phosphate acetyltransferase-N-acetylglucosamine-1-phosphate uridyltransferase, phosphofructokinase, Enzyme II^{Glc} of the PEP:glucose PTS, EIIM, P/III^{Man} of the PEP:mannose PTS, and/or alkaline phosphatase, wherein the genetic modification decreases enzymatic activity of the protein. Expression of the recombinant nucleic acid molecule increases expression of the N-glucosamine-6-phosphate synthase by the microorganism. In a further embodiment, the recombinant nucleic acid molecule is integrated into the genome of the microorganism. In yet another embodiment, the microorganism produces at least about 20 mg/L of N-glucosamine when cultured for about 24 hours at 37° C. to a cell density of at least about 8 g/L by dry cell weight, in a pH 7.0 fermentation medium comprising: 14 g/L K₂HPO₄, 16 g/L KH₂PO₄, 1 g/L Na₃Citrate.2H₂O, 5 g/L (NH₄)₂SO₄, 20 g/L glucose, 10 mM MgSO₄, 1 mM CaCl₂, and 1 mM IPTG.

### DESCRIPTION OF THE FIGURES OF THE INVENTION

FIG. 1 is a schematic representation of the pathways for the biosynthesis and catabolism of N-glucosamine and N-acetyl-glucosamine and their phosphorylated derivatives in Escherichia coli.

FIG. 2 is a schematic representation of the modifications to the pathways related to amino sugar metabolism for the overproduction of N-glucosamine in Escherichia coli.

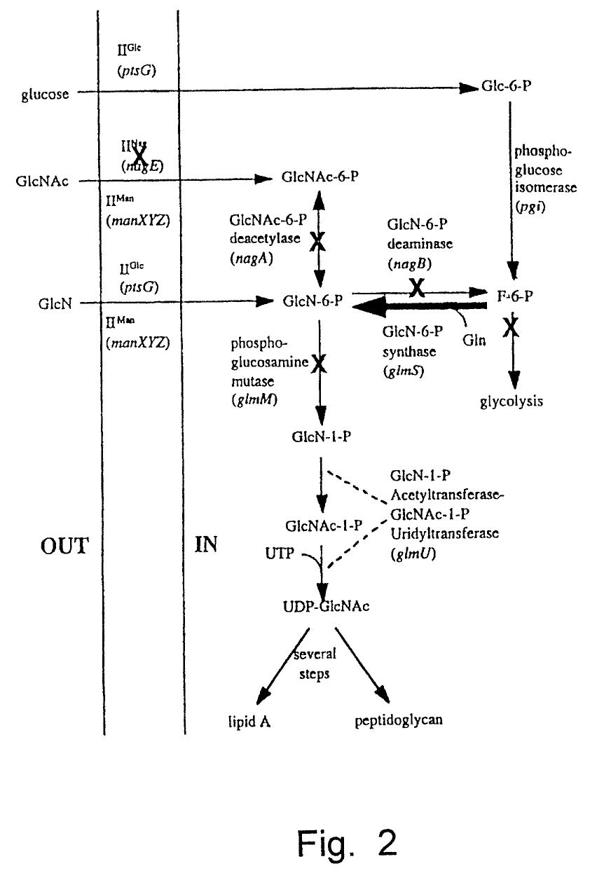

FIG. 3 is a schematic representation of the production of Escherichia coli strains containing combinations of the manXYZ, ptsG, and Δnag mutations.

FIG. 4 is a line graph illustrating the effects on N-glucosamine accumulation of feeding additional glucose and ammonium sulfate to cultures.

FIG. 5 is a line graph which shows that N-glucosamine-6-phosphate synthase is inhibited by N-glucosamine-6-phosphate and N-glucosamine.

FIG. 6 is a line graph illustrating product inhibition of N-glucosamine-6-phosphate synthase activity in mutant glmS clones.

FIG. 7 is a schematic representation of the strategy for constructions of Escherichia coli strains containing mutant glmS genes.

FIG. 8 is a line graph illustrating product inhibition of N-glucosamine-6-phosphate synthase in Escherichia coli strains with integrated mutant glmS genes.

FIG. 9 is a line graph showing N-glucosamine production in mutant Escherichia coli strains with integrated mutant glmS genes.

### DETAILED DESCRIPTION OF THE INVENTION

The present invention relates to a biosynthetic method for producing N-glucosamine. Such a method includes fermentation of a genetically modified microorganism to produce N-glucosamine. The present invention also relates to genetically modified microorganisms, such as strains of Escherichia coli, useful for producing N-glucosamine. As used herein, the terms N-glucosamine and glucosamine can be used interchangeably. Similarly, the terms N-glucosamine-6-phosphate and glucosamine-6-phosphate can be used interchangeably. N-glucosamine can also be abbreviated as GlcN and N-glucosamine-6-phosphate can also be abbreviated as GlcN-6-P.

The novel method of the present invention for production of N-glucosamine by fermentation is inexpensive and can produce a yield of N-glucosamine that exceeds the yield per cost of N-glucosamine produced by current hydrolysis methods. In addition, by using the genetically modified microorganism as described herein, the method of the present invention can be easily modified to adapt to particular problems or changing needs relative to the production of N-glucosamine.

The amino sugars, N-acetylglucosamine (GlcNAc) and N-glucosamine (GlcN), are fundamentally important molecules in microorganisms, because they are the precursors for the biosynthesis of major macromolecules, and in particular, glycoconjugates (i.e., macromolecules containing covalently bound oligosaccharide chains). For example, in Escherichia coli, N-acetylglucosamine and N-glucosamine are precursors for two macromolecules or the cell envelope, peptidoglycan and lipopolysaccharide. Mutations that block the biosynthesis of peptidoglycan or lipopolysaccharide are lethal, resulting in loss of integrity of the cell envelope and ultimately in cell lysis.

One embodiment of the present invention relates to a method to produce N-glucosamine by fermentation of a microorganism. This method includes the steps of (a) culturing in a fermentation medium a microorganism having a genetic modification in an amino sugar metabolic pathway which includes: a pathway for converting N-glucosamine-6-phosphate into another compound, a pathway for synthesizing N-glucosamine-6-phosphate, a pathway for transport of N-glucosamine or N-glucosamine-6-phosphate out of said microorganism, a pathway for transport of N-glucosamine into said microorganism, and a pathway which competes for substrates involved in the production of N-glucosamine-6-phosphate, to produce a product which can include intracellular N-glucosamine-6-phosphate and/or extracellular N-glucosamine from the microorganism; and (b) recovering the product by recovering intracellular N-glucosamine-6-phosphate from the microorganism and/or recovering extracellular N-glucosamine from the fermentation medium. The fermentation medium includes assimilable sources of carbon, nitrogen and phosphate.

Another embodiment of the present invention relates to a method to produce N-glucosamine by fermentation. Such method includes the steps of: (a) culturing in a fermentation medium comprising assimilable sources of carbon, nitrogen and phosphate, an Escherichia coli transformed with a recombinant nucleic acid molecule encoding N-glucosamine-6-phosphate synthase operatively linked to a transcription control sequence; and (b) recovering a product selected from the group of N-glucosamine-6-phosphate and N-glucosamine. The recombinant nucleic acid molecule increases expression of the N-glucosamine-6-phosphate synthase by the Escherichia coli. In a further embodiment, the recombinant nucleic acid molecule comprises a genetic modification which reduces N-glucosamine -6-phosphate product inhibition of the N-glucosamine-6-phosphate synthase. In yet another embodiment, the Escherichia coli has an additional genetic modification in at least one gene selected from the group of nagA, nagB, nagC, nagD, nagE, manXYZ, glmM, pfkB, pfkA, glmU, glmS, ptsG and/or alkaline phosphatase gene.

To produce significantly high yields of N-glucosamine by the fermentation method of the present invention, a microorganism is genetically modified to enhance production of N-glucosamine. As used herein, a genetically modified microorganism, such as Escherichia coli, has a genome which is modified (i.e., mutated or changed) from its normal (i.e., wild-type or naturally occurring) form. Genetic modification of a microorganism can be accomplished using classical strain development and/or molecular genetic techniques. Such techniques are generally disclosed, for example, in Sambrook et al., 1989, Molecular Cloning: A Laboratory Manual, Cold Spring Harbor Labs Press. The reference Sambrook et al., ibid., is incorporated by reference herein in its entirety. A genetically modified microorganism can include a natural genetic variant as well as a microorganism in which nucleic acid molecules have been inserted, deleted or modified (i.e., mutated; e.g., by insertion, deletion, substitution, and/or inversion of nucleotides), in such a manner that such modifications provide the desired effect within the microorganism. According to the present invention, a genetically modified microorganism includes a microorganism that has been modified using recombinant technology. As used herein, genetic modifications which result in a decrease in gene expression, in the function of the gene, or in the function of the gene product (i.e., the protein encoded by the gene) can be referred to as inactivation (complete or partial), deletion, interruption, blockage or down-regulation of a gene. For example, a genetic modification in a gene which results in a decrease in the function of the protein encoded by such gene, can be the result of a complete deletion of the gene (i.e., the gene does not exist, and therefore the protein does not exist), a mutation in the gene which results in incomplete or no translation of the protein (e.g., the protein is not expressed), or a mutation in the gene which decreases or abolishes the natural function of the protein (e.g., a protein is expressed which has decreased or no enzymatic activity). Genetic modifications which result in an increase in gene expression or function can be referred to as amplification, overproduction, overexpression, activation, enhancement, addition, or up-regulation of a gene.

An amino sugar is an amino derivative of a saccharide (e.g., a saccharide having an amino group in place of a hydroxyl group). According to the present invention, an amino sugar metabolic pathway is any biochemical pathway involved in, or affecting, the biosynthesis, anabolism or catabolism of an amino sugar. As used herein, amino sugar metabolic pathways include pathways involved in the transport of amino sugars and their precursors into and out of a cell, and can also include biochemical pathways which compete for substrates involved in the biosynthesis or catabolism of an amino sugar. For example, the immediate precursor to one of the earliest formed amino sugars is fructose-6-phosphate (F-6-P), which, in a biochemical reaction with glutamine (Gln, the amino group donor), forms N-glucosamine-6-phosphate. Fructose-6-phosphate is also an intermediate in the glycolysis pathway. Therefore, the glycolysis pathway competes with the N-glucosamine-6-phosphate biosynthesis pathway by competing for a substrate, fructose-6-phosphate. In addition, N-glucosamine-6-phosphate can be converted to other amino sugars and form constituents in various macromolecules by a series of biochemical reactions. As such, the fructose-6-phosphate/N-glucosamine-6-phosphate pathway, the fructose-6-phosphate glycolysis pathway, to the extent that it affects the biosynthesis of N-glucosamine-6-phosphate, and the N-glucosamine-6-phosphate/macromolecule biosynthesis pathway are all considered to be amino sugar metabolic pathways in the present invention.

In general, a microorganism having a genetically modified amino sugar metabolic pathway has at least one genetic modification, as discussed above, which results in a change in one or more amino sugar metabolic pathways as described above as compared to a wild-type microorganism cultured under the same conditions. Such a modification in an amino sugar metabolic pathway changes the ability of the microorganism to produce an amino sugar. According to the present invention, a genetically modified microorganism preferably has an enhanced ability to produce N-glucosamine compared to a wild-type microorganism cultured under the same conditions. An amino sugar metabolic pathway which affects the production of N-glucosamine can generally be categorized into at least one of the following kinds of pathways: (a) pathways for converting N-glucosamine-6-phosphate into other compounds, (b) pathways for synthesizing N-glucosamine-6-phosphate, (c) pathways for transporting N-glucosamine into a cell, (d) pathways for transporting N-glucosamine or N-glucosamine-6-phosphate out of a cell, and (e) pathways which compete for substrates involved in the production of N-glucosamine-6-phosphate.

A genetically modified microorganism useful in a method of the present invention typically has at least one modified gene involved in at least one amino sugar metabolic pathway which results in (a) reduced ability to convert N-glucosamine-6-phosphate into other compounds (i.e., inhibition of N-glucosamine-6-phosphate catabolic or anabolic pathways), (b) an enhanced ability to produce (i.e., synthesize) N-glucosamine-6-phosphate, (c) a reduced ability to transport N-glucosamine into the cell, (d) an enhanced ability to transport N-glucosamine-6-phosphate or N-glucosamine out of the cell, and/or (e) a reduced ability to use substrates involved in the production of N-glucosamine-6-P for competing biochemical reactions.

It is to be understood that the present invention discloses a method comprising the use of a microorganism with an ability to produce commercially useful amounts of N-glucosamine in a fermentation process (i.e., preferably an enhanced ability to produce N-glucosamine compared to a wild-type microorganism cultured under the same conditions). This method is achieved by the genetic modification of one or more genes encoding a protein involved in an amino sugar metabolic pathway which results in the production (expression) of a protein having an altered (e.g., increased or decreased) function as compared to the corresponding wild-type protein. Such an altered function enhances the ability of the genetically engineered microorganism to produce N-glucosamine. It will be appreciated by those of skill in the art that production of genetically modified microorganisms having a particular altered function as described elsewhere herein (e.g., an enhanced ability to produce N-glucosamine-6-phosphate) such as by the specific selection techniques described in the Examples, can produce many organisms meeting the given functional requirement, albeit by virtue of a variety of different genetic modifications. For example, different random nucleotide deletions and/or substitutions in a given nucleic acid sequence may all give rise to the same phenotypic result (e.g., decreased enzymatic activity of the protein encoded by the sequence). The present invention contemplates any such genetic modification which results in the production of a microorganism having the characteristics set forth herein.

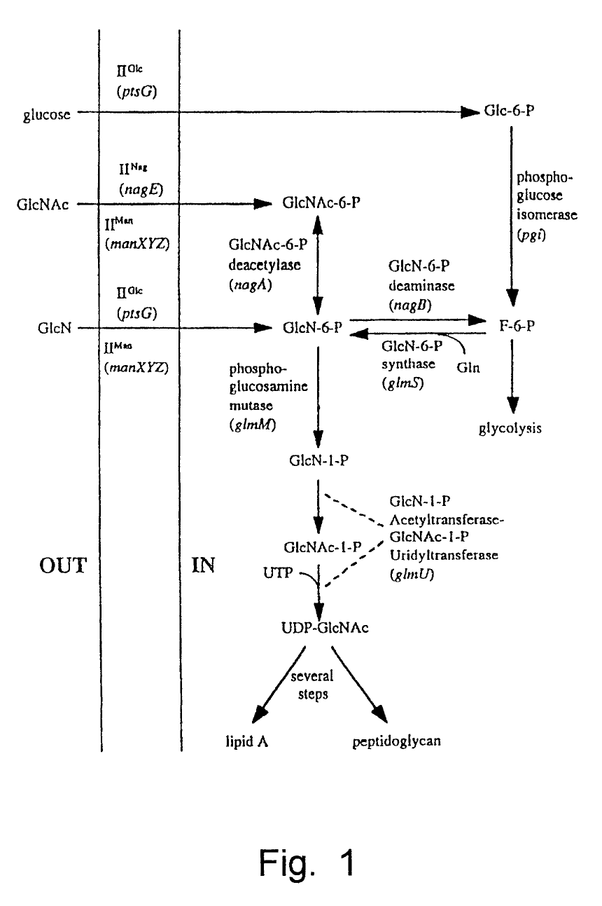

For a variety of microorganisms, many of the amino sugar metabolic pathways have been elucidated. In particular, all of the pathways for the biosynthesis and catabolism of N-glucosamine and N-acetylglucosamine and their phosphorylated derivatives have been elucidated in Escherichia coli. These pathways include the multiple transport systems for the utilization of these amino sugars as carbon sources. All of the genes encoding the enzymes and proteins directly related to the transport, catabolism and biosynthesis of amino sugars in Escherichia coli have been cloned and sequenced. In addition, mutant strains of Escherichia coli blocked in substantially every step of amino sugar metabolism have been isolated. The pathways for amino sugar metabolism for Escherichia coli are illustrated in FIG. 1.

As will be discussed in detail below, even though many of the pathways and genes involved in the amino sugar metabolic pathways have been elucidated, until the present invention, it was not known which of the many possible genetic modifications would be necessary to generate a microorganism that can produce commercially significant amounts of N-glucosamine. Indeed, the present inventors are the first to design and engineer an N-glucosamine-producing microorganism that has N-glucosamine production capabilities that far exceed the N-glucosamine production capability of any known wild-type or mutant microorganism. The present inventors are also the first to appreciate that such a genetically modified microorganism is useful in a method to produce N-glucosamine for commercial use.

A microorganism to be used in the fermentation method of the present invention is preferably a bacterium or a yeast. More preferably, such a microorganism is a bacterium of the genus Escherichia. Escherichia coli is the most preferred microorganism to use in the fermentation method of the present invention. Particularly preferred strains of Escherichia coli include K-12, B and W, and most preferably, K-12. Although Escherichia coli is most preferred, it is to be understood that any microorganism that produces N-glucosamine and can be genetically modified to enhance production of N-glucosamine can be used in the method of the present invention. A microorganism for use in the fermentation method of the present invention can also be referred to as a production organism.

The amino sugar metabolic pathways of the microorganism, Escherichia coli, will be addressed as specific embodiments of the present invention are described below. It will be appreciated that other microorganisms and in particular, other bacteria, have similar amino sugar metabolic pathways and genes and proteins having similar structure and function within such pathways. As such, the principles discussed below with regard to Escherichia coli are applicable to other microorganisms.

In one embodiment of the present invention, a genetically modified microorganism includes a microorganism which has an enhanced ability to synthesize N-glucosamine-6-phosphate. According to the present invention, “an enhanced ability to synthesize” a product refers to any enhancement, or up-regulation, in an amino sugar metabolic pathway related to the synthesis of the product such that the microorganism produces an increased amount of the product compared to the wild-type microorganism cultured under the same conditions. In one embodiment of the present invention, enhancement of the ability of a microorganism to synthesize N-glucosamine-6-phosphate is accomplished by amplification of the expression of the glucose-6-phosphate synthase gene, which in Escherichia coli is the glmS gene, the product of which is N-glucosamine-6-phosphate synthase. N-glucosamine-6-phosphate synthase catalyzes the reaction in which fructose-6-phosphate and glutamine form N-glucosamine-6-phosphate. Amplification of the expression of N-glucosamine-6-phosphate synthase can be accomplished in Escherichia coli, for example, by introduction of a recombinant nucleic acid molecule encoding the glmS gene.

Overexpression of glmS is crucial for the intracellular accumulation of N-glucosamine-6-phosphate and ultimately for production of N-glucosamine, since the level of N-glucosamine-6-phosphate synthase in the cell will control the redirection of carbon flow away from glycolysis and into N-glucosamine-6-phosphate synthesis. The glmS gene is located at 84 min on the Escherichia coli chromosome, and sequence analysis of this region of the chromosome reveals that glmS resides in an operon with the glmU gene, which encodes the bifunctional enzyme, N-glucosamine-1-phosphate acetyltransferase-N-acetylglucosamine-1-phosphate uridyltransferase. N-glucosamine-1-phosphate acetyltransferase-N-acetylglucosamine-1-phosphate uridyltransferase functions within the amino sugar metabolic pathway in which N-glucosamine-6-phosphate is incorporated, through a series of biochemical reactions, into macromolecules. No obvious promoter sequence is detected upstream of glmS; transcription of the glmUS operon is initiated from two promoter sequences upstream of glmU. Thus, it is preferred that the glmS gene be cloned under control of an artificial promoter. The promoter can be any suitable promoter that will provide a level of glmS expression required to maintain a sufficient level of N-glucosamine-6-phosphate synthase in the production organism. Preferred promoters are constitutive (rather than inducible) promoters, since the need for addition of expensive inducers is therefore obviated. Particularly preferred promoters to be used with glmS are lac and λPL. The gene dosage (copy number) of glmS can be varied according to the requirements for maximum product formation. In one embodiment, the recombinant glmS gene is integrated into the E. coli chromosome.

The reported Kₘ's of N-glucosamine-6-phosphate synthase from Escherichia coli are 2 mM and 0.4 mM for fructose-6-phosphate and glutamine, respectively. These are relatively high values (i.e., the affinity of the enzyme for its substrates is rather weak). It is therefore another embodiment of the present invention to provide a microorganism having a N-glucosamine-6-phosphate synthase with improved affinity for its substrates. A N-glucosamine-6-phosphate synthase with improved affinity for its substrates can be produced by any suitable method of genetic modification or protein engineering. For example, computer-based protein engineering can be used to design a N-glucosamine-6-phosphate synthase protein with greater stability and better affinity for its substrate. See for example, Maulik et al., 1997, Molecular Biotechnology: Therapeutic Applications and Strategies, Wiley-Liss, Inc., which is incorporated herein by reference in its entirety.

White (1968, Biochem. J., 106:847-858) first demonstrated that N-glucosamine-6-phosphate synthase was inhibited by N-glucosamine-6-phosphate. The present inventors determined that this inhibition was a key factor which limits N-glucosamine accumulation in N-glucosamine production strains of the present invention, which have been designed for commercial use. Therefore, it is yet another embodiment of the present invention to provide a microorganism having an N-glucosamine-6-phosphate synthase with reduced N-glucosamine-6-phosphate product feedback inhibition. An N-glucosamine-6-phosphate synthase with reduced product inhibition can be a mutated (i.e., genetically modified) N-glucosamine-6-phosphate synthase gene, for example, and can be produced by any suitable method of genetic modification. For example, a recombinant nucleic acid molecule encoding N-glucosamine-6-phosphate synthase can be modified by any method for inserting, deleting, and/or substituting nucleotides, such as by error-prone PCR. In this method, the gene is amplified under conditions that lead to a high frequency of misincorporation errors by the DNA polymerase used for the amplification. As a result, a high frequency of mutations are obtained in the PCR products. This method is described in detail in Example 5. The resulting N-glucosamine-6-phosphate synthase gene mutants can then be screened for reduced product inhibition by testing the mutant genes for the ability to confer increased N-glucosamine production onto a test microorganism, as compared to a microorganism carrying the non-mutated recombinant N-glucosamine-6-phosphate synthase nucleic acid molecule.

An adequate intracellular supply of glutamine (Gln) is critical for the N-glucosamine-6-phosphate synthase reaction. Inspection of the synthetic and degradative pathways for N-glucosamine-6-phosphate reveals the presence of a potential futile cycle whereby continuous interconversion of fructose-6-phosphate and N-glucosamine-6-phosphate results in wasteful depletion of glutamine. In one embodiment of the present invention, the supply of glutamine can be increased either by genetic modification of the production organism to increase glutamine production in the cell, or by modifying the fermentation medium (i.e., adding glutamine to the fermentation medium), to ensure that the supply of glutamine will not limit the production of N-glucosamine-6-phosphate.

In another embodiment of the present invention, the potential futile cycling of fructose-6-phosphate and N-glucosamine-6-phosphate is addressed by inhibiting, or blocking, the reverse reaction in which N-glucosamine-6-phosphate is converted into fructose-6-phosphate. In this embodiment, a microorganism is genetically modified to have an inactivation or deletion of the gene which catalyzes this conversion, N-glucosamine-6-phosphate deaminase, which in Escherichia coli is the nagB gene. nagB is one of several nag genes which are part of the nag regulon. The nag genes involved in the degradation of N-glucosamine and N-acetyl-glucosamine exist as a regulon located at 15 min on the Escherichia coli chromosome. In another embodiment, the entire nag regulon is inactivated or deleted. The advantages of deleting the entire nag regulon are discussed in detail below.

As discussed above, overproduction of N-glucosamine-6-phosphate synthase results in diversion of fructose-6-phosphate synthesis to N-glucosamine-6-phosphate synthesis. However, many other enzymes can compete for the substrate, fructose-6-phosphate. Therefore, one embodiment of the present invention includes a microorganism in which these competitive side reactions are blocked. In a preferred embodiment, a microorganism having complete or partial inactivation of the gene encoding phosphofructokinase is provided. The second step in the glycolytic pathway is the conversion of fructose-6-phosphate to fructose-1,6-diphosphate by phosphofructokinase, which in Escherichia coli exists as two isozymes encoded by the pfkA and pfkB genes. Complete or partial inactivation of either the pfkA or pfkB genes slows the entry of fructose-6-phosphate into the glycolytic pathway and enhances the conversion of fructose-6-phosphate to N-glucosamine-6-phosphate. As used herein, inactivation of a gene can refer to any modification of a gene which results in a decrease in the activity (i.e., expression or function) of such a gene, including attenuation of activity or complete deletion of activity.

In a further embodiment of the present invention, a genetically modified microorganism has a decreased ability to convert N-glucosamine-6-phosphate into other compounds. Inactivation of N-glucosamine-6-phosphate deaminase, as described above, represents one such modification, however, N-glucosamine-6-phosphate serves as a substrate for other biochemical reactions. The first committed step in the pathway leading to production of macromolecules such as lipopolysaccharide and peptidoglycan in Escherichia coli is the conversion of N-glucosamine-6-phosphate to N-glucosamine-1-phosphate by phosphoglucosamine mutase, which in Escherichia coli is the product of the glmM gene. The involvement of this enzyme activity in the pathway of lipopolysaccharide and peptidoglycan biosynthesis was recently confirmed with the cloning of the glmM gene. Consequently, the regulation of glmM gene, and its cognate product, phosphoglucosamine mutase, has not been studied in detail. It has been shown, however, that the phosphoglucosamine mutase, like all other hexosephosphate mutase enzymes studied, is regulated by phosphorylation. This type of regulation at the enzyme level is typically exquisitely sensitive to levels of the pathway end products. Thus, carbon flow through phosphoglucosamine mutase can be self-regulating and may not be a problem as N-glucosamine-6-phosphate accumulates. Since the sequence of the glmM gene is known, however, it is a preferred embodiment of the present invention to provide a microorganism in which the gene encoding phosphoglucosamine mutase is interrupted or deleted. More preferably, the gene encoding phosphoglucosamine mutase is down-regulated, but not completely inactivated, by a mutation, so as not to completely block the biosynthesis of the critical cell envelope components.

Another pathway which results in the conversion of N-glucosamine-6-phosphate to another compound is catalyzed by the enzyme, N-acetylglucosamine-6-phosphate deacetylase. N-acetylglucosamine-6-phosphate deacetylase is capable of catalyzing the reverse reaction of converting N-glucosamine-6-phosphate (plus acetyl CoA) to N-acetyl-glucosamine-6-phosphate. This could result in futile cycling of N-glucosamine-6-phosphate and N-acetyl-glucosamine-6-phosphate and result in a product composed of a mixture of N-glucosamine and N-acetyl-glucosamine. Therefore, it is a further embodiment of the present invention to provide a genetically modified microorganism in which the gene encoding N-acetylglucosamine-6-phosphate deacetylase, which is the nagA gene in Escherichia coli, is inactivated.

It is a further embodiment of the present invention to inactivate the transport systems for N-glucosamine in a microorganism such that once the N-glucosamine is excreted by the cell it is not taken back up. This modification is helpful for avoiding a high intracellular level of N-glucosamine which could be toxic to the cells, and facilitates recovery of the product, since the product remains extracellular. In a preferred embodiment of the present invention, the transportation systems for N-glucosamine are inactivated to keep N-glucosamine outside of the microorganism once it is excreted by the microorganism. During growth of Escherichia coli on N-glucosamine as sole carbon source, N-glucosamine is transported into the cell by the PEP:mannose phosphotransferase (PTS) system, which is not only capable of transporting N-glucosamine into the cell, but is also induced by N-glucosamine. It is therefore an embodiment of the present invention to provide a microorganism lacking the ability to transport N-glucosamine into the cell. For example, a manXYZ mutant (i.e., an Escherichia coli lacking or having a mutation in the genes encoding EIIM, P/III^{Man} of the PEP:mannose PTS) can not transport N-glucosamine into the cell by this mechanism. The PEP:glucose PTS of Escherichia coli, on the other hand, is capable of transporting both glucose and N-glucosamine into the cell, but N-glucosamine cannot induce this system. Thus, in order to grow a manXYZ mutant on N-glucosamine, the cells must first be grown on glucose to induce expression of the (alternate) glucose transport system and allow glucose (the preferred carbon source) to be transported into the cell. These induced cells are then capable of transporting N-glucosamine into the cell via the glucose transporter. A similar situation exists for transport of N-glucosamine by the PEP:fructose PTS, although in this case N-glucosamine transport by the enzyme II^{Fru} is poor. Methods to inhibit these secondary N-glucosamine transport pathways are discussed below. It is yet another embodiment of the present invention to provide a microorganism having a decreased function in the PEP:glucose PTS (described above). Such a modification may be necessary to avoid “reabsorption of glucosamine from the culture medium. For example, a ptsG mutant (i.e., an Escherichia coli lacking or having a mutation in the genes encoding enzyme II^{Glc} of the PEP:glucose PTS). Since such microorganisms will have reduced ability to grow using glucose as a carbon source, such organisms can be further genetically modified to take up glucose by a PEP:glucose PTS-independent mechanism. It is has been shown, for example, that mutant microorganisms can be selected which are defective in the PEP:glucose PTS and still have an ability to grow on glucose (Flores et al., 1996, Nature Biotechnology 14:620-623).

DNA sequencing of the nag regulon in Escherichia coli reveals that the nagE gene, encoding the N-acetyl-glucosamine-specific enzyme II^{Nag} protein of the PEP:sugar phosphotransferase (PTS) system, which is involved in N-glucosamine transport into the cell, resides on one arm of the regulon and is transcribed divergently from the other nag genes (nagBACD) located on the other arm of the regulon. Therefore, another genetic modification that would result in decreased ability of an Escherichia coli to transport N-glucosamine into the cell is an inactivation or deletion of the nagE gene, or a gene encoding a similar enzyme in any microorganism used in a method of the present invention.

As discussed above, in one embodiment of the present invention, a genetically modified Escherichia coli microorganism useful in a method of the present invention has a deletion of the entire nag regulon. Deletion of the entire chromosomal nag regulon is preferred, because many genes which are deleterious to the production of N-glucosamine-6-phosphate are inactivated together. The genes, nagA, nagB and nagE, have been discussed in detail above. The nagC gene encodes a regulatory protein that acts as a repressor of the nag regulon as well as both an activator and repressor of the glmUS operon. The glm genes are discussed in detail above. The function of the nagD gene is not known, but is believed to be related to amino sugar metabolism as it resides within the nag regulon. Thus, in Escherichia coli, a complete deletion of the nag regulon avoids catabolism of the initial intracellular product (N-glucosamine-6-phosphate) in a strain of Escherichia coli designed to overproduce N-glucosamine. A preferred Escherichia coli mutant strain having a deletion of the nag regulon is an Escherichia coli having a ΔnagEBACD: :tc deletion/insertion.

With regard to activation of the glmUS operon (a function of nagC), although activation of the glmS gene, encoding N-glucosamine-6-phosphate synthase, is desirable, an increase in the level of the glmU gene product, N-glucosamine-1-phosphate acetyltransferase-N-acetylglucosamine-1-phosphate uridyltransferase could be deleterious to accumulation of N-glucosamine-6-phosphate as it could lead to siphoning off of carbon flow toward cell envelope components. It is therefore an embodiment of the present invention to inactivate N-glucosamine-1-phosphate acetyltransferase-N-acetylglucosamine-1-phosphate uridyltransferase in a microorganism useful in a method of the present invention. In a microorganism in which the glmUS operon, or its equivalent, has been inactivated or deleted, it is a further embodiment of the present invention to genetically modify the microorganism by recombinantly producing the gene encoding N-glucosamine-6-phosphate synthase under control of an artificial promoter in the microorganism.

The initial intracellular product in the genetically modified microorganism described herein is N-glucosamine-6-phosphate. In many microorganisms, including Escherichia coli, N-glucosamine-6-phosphate is typically dephosphorylated to N-glucosamine prior to transport out of the cell. Nonetheless, it is yet another embodiment of the present invention to provide a microorganism which is genetically modified to have a suitable phosphatase activity for the conversion of N-glucosamine-6-phosphate to N-glucosamine. In a preferred embodiment, such an Escherichia coli has an enhanced level of alkaline phosphatase activity.

As noted above, in the method for production of N-glucosamine of the present invention, a microorganism having a genetically modified amino sugar metabolic pathway is cultured in a fermentation medium for production of N-glucosamine. An appropriate, or effective, fermentation medium refers to any medium in which a genetically modified microorganism of the present invention, when cultured, is capable of producing N-glucosamine. Such a medium is typically an aqueous medium comprising assimilable carbon, nitrogen and phosphate sources. Such a medium can also include appropriate salts, minerals, metals and other nutrients. One advantage of the genetic modifications to a microorganism described herein is that although such genetic modifications significantly alter the metabolism of amino sugars, they do not create any nutritional requirements for the production organism. Thus, a minimal-salts medium containing glucose as the sole carbon source is preferably used as the fermentation medium. The use of a minimal-salts-glucose medium for the N-glucosamine fermentation will also facilitate recovery and purification of the N-glucosamine product.

Microorganisms of the present invention can be cultured in conventional fermentation bioreactors. The microorganisms can be cultured by any fermentation process which includes, but is not limited to, batch, fed-batch, cell recycle, and continuous fermentation. Preferably, microorganisms of the present invention are grown by batch or fed-batch fermentation processes.

Before inoculation, the fermentation medium is brought up to the desired temperature, typically from about 25° C. to about 40° C., preferably from about 30° C. to about 40° C., and most preferably about 37° C. The medium is inoculated with an actively growing culture of the genetically modified microorganism in an amount sufficient to produce, after a reasonable growth period, a high cell density. The cells are grown to a cell density of at least about 10 g/l, preferably between about 10 g/l and about 40 g/l, and more preferably at least about 40 g/l. This process typically requires about 12 hours.

Sufficient oxygen must be added to the medium during the course of the fermentation to maintain cell growth during the initial cell growth and to maintain metabolism and N-glucosamine production. Oxygen is conveniently provided by agitation and aeration of the medium. Conventional methods, such as stirring or shaking, may be used to agitate and aerate the medium. Preferably the oxygen concentration in the medium is greater than about 15% of the saturation value (i.e., the solubility of oxygen in the medium at atmospheric pressure and about 30-40° C.) and more preferably greater than about 20% of the saturation value, although excursions to lower concentrations may occur if fermentation is not adversely affected. The oxygen concentration of the medium can be monitored by conventional methods, such as with an oxygen probe electrode. Other sources of oxygen, such as undiluted oxygen gas and oxygen gas diluted with inert gas other than nitrogen, can be used.

Since the production of N-glucosamine by fermentation is preferably based on using glucose as the sole carbon source, in a preferred embodiment, in Escherichia coli, the PEP:glucose PTS will be induced. Accordingly, even in the absence of a functional EIIM, P/III^{Man} of the PEP:mannose PTS (e.g., in an Escherichia coli having a manXYZ mutation), the product, N-glucosamine, will still be taken up by the cells via the induced glucose transport system. In the presence of excess glucose, however, uptake of N-glucosamine is severely repressed. Thus, it is one embodiment of the present invention to prevent uptake of the N-glucosamine product by maintaining an excess of glucose in the fermentation bioreactor. As used herein, “an excess” of glucose refers to an amount of glucose above that which is required to maintain the growth of the microorganism under normal conditions. Preferably, the glucose concentration is maintained at a concentration of from about 0.5% to about 5% weight/volume of the fermentation medium. In another embodiment, the glucose concentration is maintained at a concentration of from about 5 g/L to about 50 g/L of the fermentation medium, and even more preferably, from about 5 g/L to about 20 g/L of the fermentation medium. In one embodiment, the glucose concentration of the fermentation medium is monitored by any suitable method (e.g., by using glucose test strips), and when the glucose concentration is at or near depletion, additional glucose can be added to the medium. In another embodiment, the glucose concentration is maintained by semi-continuous or continuous feeding of the fermentation medium. The parameters disclosed herein for glucose can be applied to any carbon source used in the fermentation medium of the present invention.

It is a further embodiment of the present invention to supplement and/or control other components and parameters of the fermentation medium, as necessary to maintain and/or enhance the production of N-glucosamine by a production organism. For example, in one embodiment, the fermentation medium includes ammonium sulfate, and the ammonium sulfate concentration in the culture medium is supplemented by the addition of excess ammonium sulfate. Preferably, the amount of ammonium sulfate is maintained at a level of from about 0.1% to about 1% (weight/volume) in the fermentation medium, and preferably, at about 0.5%. In yet another embodiment, the pH of the fermentation medium is monitored for fluctuations in pH. In the fermentation method of the present invention, the pH is preferably maintained at a pH of from about pH 6.0 to about pH 8.0, and more preferably, at about pH 7.0. In the method of the present invention, if the starting pH of the fermentation medium is pH 7.0, the pH of the fermentation medium is monitored for significant variations from pH 7.0, and is adjusted accordingly, for example, by the addition of sodium hydroxide.

A further embodiment of the present invention is to redirect carbon flux from acetate production to the production of less toxic byproducts. By such methods, problems of toxicity associated with an excess of glucose in the fermentation medium can be avoided. Methods to redirect carbon flux from acetate production are known in the art.

In a batch fermentation process of the present invention, fermentation is continued until the formation of N-glucosamine, as evidenced by the accumulation of extracellular N-glucosamine, essentially ceases. The total fermentation time is typically from about 40 to about 60 hours, and more preferably, about 48 hours. In a continuous fermentation process, N-glucosamine can be removed from the bioreactor as it accumulates in the medium. The method of the present invention results in production of a product which can include intracellular or extracellular N-glucosamine-6-phosphate and intracellular or extracellular N-glucosamine.

The method of the present invention further includes recovering the product, which can be intracellular N-glucosamine-6-phosphate or extracellular N-glucosamine. The phrase “recovering N-glucosamine” refers simply to collecting the product from the fermentation bioreactor and need not imply additional steps of separation or purification. For example, the step of recovering can refer to removing the entire culture (i.e., the microorganism and the fermentation medium) from the bioreactor, removing the fermentation medium containing extracellular N-glucosamine from the bioreactor, and/or removing the microorganism containing intracellular N-glucosamine-6-phosphate from the bioreactor. These steps can be followed by further purification steps. N-glucosamine is preferably recovered in substantially pure form. As used herein, “substantially pure” refers to a purity that allows for the effective use of the N-glucosamine as a nutriceutical compound for commercial sale. In one embodiment, the N-glucosamine product is preferably separated from the production organism and other fermentation medium constituents. Methods to accomplish such separation are described below.

Typically, most of the N-glucosamine produced in the present process is extracellular. The microorganism can be removed from the fermentation medium by conventional methods, such as by filtration or centrifugation. In one embodiment, the step of recovering the product includes the purification of N-glucosamine from the fermentation medium. N-glucosamine can be recovered from the cell-free fermentation medium by conventional methods, such as, ion exchange, chromatography, extraction, crystallization (e.g., evaporative crystallization), membrane separation, reverse osmosis and distillation. In a preferred embodiment, N-glucosamine is recovered from the cell-free fermentation medium by crystallization. In another embodiment, the step of recovering the product includes the step of concentrating the extracellular N-glucosamine.

In one embodiment, N-glucosamine-6-phosphate accumulates intracellularly, the step of recovering the product includes isolating N-glucosamine-6-phosphate from the microorganism. For example, the product can be recovered by lysing the microorganism cells by a method which does not degrade the N-glucosamine product, centrifuging the lysate to remove insoluble cellular debris, and then recovering the N-glucosamine and/or N-glucosamine-6-phosphate product by a conventional method as described above.

The initial intracellular product in the genetically modified microorganism described herein is N-glucosamine-6-phosphate. It is generally accepted that phosphorylated intermediates are dephosphorylated during export from the microorganism, most likely due to the presence of several phosphatases in the periplasmic space of the microorganism. In one embodiment of the present invention, N-glucosamine-6-phosphate is dephosphorylated before or during export from the cell by naturally occurring phosphatases in order to facilitate the production of the desired product, N-glucosamine. In this embodiment, the need for amplification of a recombinantly provided phosphatase activity in the cell or treatment of the fermentation medium with a phosphatase is obviated. In another embodiment, the level of alkaline phosphatase in the production organism is increased by a method including, but not limited to, genetic modification of the endogenous alkaline phosphatase gene or by recombinant modification of the microorganism to express an alkaline phosphatase gene. In yet another embodiment, the recovered fermentation medium is treated with a phosphatase after N-glucosamine-6-phosphate is released into the medium, such as when cells are lysed as described above.

As noted above, the process of the present invention produces significant amounts of extracellular N-glucosamine. In particular, the process produces extracellular N-glucosamine such that greater than about 50% of total N-glucosamine is extracellular, more preferably greater than about 75% of total N-glucosamine is extracellular, and most preferably greater than about 90% of total N-glucosamine is extracellular. By the method of the present invention, production of an extracellular N-glucosamine concentration can be achieved which is greater than about 1 g/l, more preferably greater than about 5 g/l, even more preferably greater than about 10 g/l, and more preferably greater than about 50 g/l.

One embodiment of the present invention relates to a method to produce N-glucosamine by fermentation which includes the steps of (a) culturing an Escherichia coli having a genetically modified amino sugar metabolic pathway in a fermentation medium comprising assimilable sources of carbon, nitrogen and phosphate to produce a product, and (b) recovering the product. The product includes intracellular N-glucosamine-6-phosphate which is recovered from the Escherichia coli and/or extracellular N-glucosamine which is recovered from the fermentation medium.

One embodiment of the present invention relates to a microorganism for producing N-glucosamine by a biosynthetic process. The microorganism is transformed with a recombinant nucleic acid molecule encoding N-glucosamine-6-phosphate synthase operatively linked to a transcription control sequence. The recombinant nucleic acid molecule has a genetic modification which reduces N-glucosamine-6-phosphate product inhibition of the N-glucosamine-6-phosphate synthase. Expression of the recombinant nucleic acid molecule increases expression of the N-glucosamine-6-phosphate synthase by the microorganism. In a preferred embodiment, the recombinant nucleic acid molecule is integrated into the genome of the microorganism. In a further embodiment, the microorganism has at least one additional genetic modification in a gene encoding a protein selected from the group of N-acetylglucosamine-6-phosphate deacetylase, N-glucosamine-6-phosphate deaminase, N-acetyl-glucosamine-specific enzyme II^{Nag}, phosphoglucosamine mutase, N-glucosamine-1-phosphate acetyltransferase-N-acetylglucosamine-1-phosphate uridyltransferase, phosphofructokinase, Enzyme II^{Glc} of the PEP:glucose PTS, EIIM, P/III^{Man} of the PEP:mannose PTS, and/or alkaline phosphatase. The genetic modification decreases the enzymatic activity of the protein. In another preferred embodiment, the microorganism has a modification in genes encoding N-acetylglucosamine-6-phosphate deacetylase, N-glucosamine-6-phosphate deaminase and N-acetyl-glucosamine-specific enzyme II^{Nag}, wherein the genetic modification decreases enzymatic activity of the protein. In one embodiment, the genetic modification is a deletion of at least a portion of the genes.

In a preferred embodiment, the genetically modified microorganism is a bacterium or a yeast, and more preferably, a bacterium of the genus Escherichia, and even more preferably, Escherichia coli. A genetically modified Escherichia coli preferably has a modification in a gene which includes, but is not limited to, nagA, nagB, nagC, nagD, nagE, manXYZ, glmM, pfkB, pfkA, glmU, glmS, ptsG or alkaline phosphatase gene. In another embodiment, such a genetically modified Escherichia coli has a deletion of nag regulon genes, and in yet another embodiment, a deletion of nag regulon genes and a genetic modification in manXYZ genes such that the proteins encoded by the manXYZ genes have decreased enzymatic activity.

Yet another embodiment of the present invention relates to a microorganism for producing N-glucosamine by a biosynthetic process which has a recombinant nucleic acid molecule encoding N-glucosamine-6-phosphate synthase operatively linked to a transcription control sequence; and at least one genetic modification in a gene encoding a protein selected from the group of N-acetylglucosamine-6-phosphate deacetylase, N-glucosamine-6-phosphate deaminase, N-acetyl-glucosamine-specific enzyme II^{Nag}, phosphoglucosamine mutase, N-glucosamine-1-phosphate acetyltransferase-N-acetylglucosamine-1-phosphate uridyltransferase, phosphofructokinase, Enzyme II^{Glc} of the PEP:glucose PTS, EIIM, P/III^{Man} of the PEP:mannose PTS, and/or alkaline phosphatase. The genetic modification decreases enzymatic activity of said protein and expression of the recombinant nucleic acid molecule increases expression of the N-glucosamine-6-phosphate synthase by the microorganism. In a preferred embodiment, the recombinant nucleic acid molecule is integrated into the genome of the microorganism.

Another embodiment of the present invention relates to any of the above-described microorganisms which produces at least about 1 g/L of N-glucosamine when cultured for about 24 hours at 37° C. to a cell density of at least about 8 g/L by dry cell weight, in a pH 7.0 fermentation medium comprising: 14 g/L K₂HPO₄, 16 g/L KH₂PO₄, 1 g/L Na₃Citrate.2H₂O, 5 g/L (NH₄)₂SO₄, 20 g/L glucose, 10 mM MgSO₄, 1 mM CaCl₂, and 1 mM IPTG.

A number of specific microorganisms are identified in the Examples section. Additional embodiments of the present invention include these microorganisms and microorganisms having the identifying characteristics of the microorganisms specifically identified in the Examples. Such microorganisms are preferably yeast or bacteria, more preferably, are bacteria, and most preferably are E. coli. Such identifying characteristics can include any or all genotypic and/or phenotypic characteristics of the microorganisms in the Examples, including their abilities to produce N-glucosamine.

Development of a microorganism with enhanced ability to produce N-glucosamine by genetic modification can be accomplished using both classical strain development and molecular genetic techniques. In general, the strategy for creating a microorganism with enhanced N-glucosamine production is to (1) inactivate or delete at least one, and preferably more than one of the amino sugar metabolic pathways in which production of N-glucosamine-6-phosphate is negatively affected (e.g., inhibited), and (2) amplify at least one, and preferably more than one of the amino sugar metabolic pathways in which N-glucosamine-6-phosphate production is enhanced. As such, genetically modified microorganisms of the present invention have a (a) reduced ability to convert N-glucosamine-6-phosphate into other compounds (i.e., inhibition of N-glucosamine-6-phosphate catabolic or anabolic pathways), (b) an enhanced ability to produce (i.e., synthesize) N-glucosamine-6-phosphate, (c) a reduced ability to transport N-glucosamine into the cell, (d) an enhanced ability to transport N-glucosamine-6-phosphate or N-glucosamine out of the cell, and/or (e) a reduced ability to use substrates involved in the production of N-glucosamine-6-P for competing biochemical reactions.

As previously discussed herein, in one embodiment, a genetically modified microorganism can be a microorganism in which nucleic acid molecules have been deleted, inserted or modified, such as by insertion, deletion, substitution, and/or inversion of nucleotides, in such a manner that such modifications provide the desired effect within the microorganism. A genetically modified microorganism can be modified by recombinant technology, such as by introduction of an isolated nucleic acid molecule into a microorganism. For example, a genetically modified microorganism can be transfected with a recombinant nucleic acid molecule encoding a protein of interest, such as a protein for which increased expression is desired. The transfected nucleic acid molecule can remain extrachromosomal or can integrate into one or more sites within a chromosome of the transfected (i.e., recombinant) host cell in such a manner that its ability to be expressed is retained. Preferably, once a host cell of the present invention is transfected with a nucleic acid molecule, the nucleic acid molecule is integrated into the host cell genome. A significant advantage of integration is that the nucleic acid molecule is stably maintained in the cell. In a preferred embodiment, the integrated nucleic acid molecule is operatively linked to a transcription control sequence (described below) which can be induced to control expression of the nucleic acid molecule.

A nucleic acid molecule can be integrated into the genome of the host cell either by random or targeted integration. Such methods of integration are known in the art. For example, as described in detail in Example 2, E. coli strain ATCC 47002 (Table 1) contains mutations that confer upon it an inability to maintain plasmids which contain a ColE1 origin of replication. When such plasmids are transferred to this strain, selection for genetic markers contained on the plasmid results in integration of the plasmid into the chromosome. This strain can be transformed, for example, with plasmids containing the gene of interest and a selectable marker flanked by the 5′-and 3′-termini of the E. coli lacZ gene. The lacZ sequences target the incoming DNA to the lacZ gene contained in the chromosome. Integration at the lacZ locus replaces the intact lacZ gene, which encodes the enzyme β-galactosidase, with a partial lacZ gene interrupted by the gene of interest. Successful integrants can be selected for β-galactosidase negativity. A genetically modified microorganism can also be produced by introducing nucleic acid molecules into a recipient cell genome by a method such as by using a transducing bacteriophage. The use of recombinant technology and transducing bacteriophage technology to produce several different genetically modified microorganism of the present invention is known in the art and is described in detail in the Examples section.

According to the present invention, a gene, for example the pstG gene, includes all nucleic acid sequences related to a natural pstG gene such as regulatory regions that control production of the pstG protein (Enzyme II^{Glc} of the PEP:glucose PTS) encoded by that gene (such as, but not limited to, transcription, translation or post-translation control regions) as well as the coding region itself. In another embodiment, a gene, for example the pstG gene, can be an allelic variant that includes a similar but not identical sequence to the nucleic acid sequence encoding a given pstG gene. An allelic variant of a pstG gene which has a given nucleic acid sequence is a gene that occurs at essentially the same locus (or loci) in the genome as the gene having the given nucleic acid sequence, but which, due to natural variations caused by, for example, mutation or recombination, has a similar but not identical sequence. Allelic variants typically encode proteins having similar activity to that of the protein encoded by the gene to which they are being compared. Allelic variants can also comprise alterations in the 5′ or 3′ untranslated regions of the gene (e.g., in regulatory control regions). Allelic variants are well known to those skilled in the art and would be expected to be found within a given microorganism, such as an E. coli, and/or among a group of two or more microorganisms.

In accordance with the present invention, an isolated nucleic acid molecule is a nucleic acid molecule that has been removed from its natural milieu (i.e., that has been subject to human manipulation). As such, “isolated” does not reflect the extent to which the nucleic acid molecule has been purified. An isolated nucleic acid molecule can include DNA, RNA, or derivatives of either DNA or RNA. There is no limit, other than a practical limit, on the maximal size of a nucleic acid molecule in that the nucleic acid molecule can include a portion of a gene, an entire gene, or multiple genes, or portions thereof.

An isolated nucleic acid molecule of the present invention can be obtained from its natural source either as an entire (i.e., complete) gene or a portion thereof capable of forming a stable hybrid with that gene. An isolated nucleic acid molecule can also be produced using recombinant DNA technology (e.g., polymerase chain reaction (PCR) amplification, cloning) or chemical synthesis. Isolated nucleic acid molecules include natural nucleic acid molecules and homologues thereof, including, but not limited to, natural allelic variants and modified nucleic acid molecules in which nucleotides have been inserted, deleted, substituted, and/or inverted in such a manner that such modifications provide the desired effect within the microorganism.

A nucleic acid molecule homologue can be produced using a number of methods known to those skilled in the art (see, for example, Sambrook et al., ibid.). For example, nucleic acid molecules can be modified using a variety of techniques including, but not limited to, classic mutagenesis techniques and recombinant DNA techniques, such as site-directed mutagenesis, chemical treatment of a nucleic acid molecule to induce mutations, restriction enzyme cleavage of a nucleic acid fragment, ligation of nucleic acid fragments, PCR amplification and/or mutagenesis of selected regions of a nucleic acid sequence, synthesis of oligonucleotide mixtures and ligation of mixture groups to “build” a mixture of nucleic acid molecules and combinations thereof. Nucleic acid molecule homologues can be selected from a mixture of modified nucleic acids by screening for the function of the protein encoded by the nucleic acid and/or by hybridization with a wild-type gene. Examples of such techniques are described in detail in the Examples section.

Although the phrase “nucleic acid molecule” primarily refers to the physical nucleic acid molecule and the phrase “nucleic acid sequence” primarily refers to the sequence of nucleotides on the nucleic acid molecule, the two phrases can be used interchangeably, especially with respect to a nucleic acid molecule, or a nucleic acid sequence, being capable of encoding a gene involved in an amino sugar metabolic pathway.

Knowing the nucleic acid sequences of certain nucleic acid molecules of the present invention, and particularly Escherichia coli nucleic acid molecules, allows one skilled in the art to, for example, (a) make copies of those nucleic acid molecules and/or (b) obtain nucleic acid molecules including at least a portion of such nucleic acid molecules (e.g., nucleic acid molecules including full-length genes, full-length coding regions, regulatory control sequences, truncated coding regions). Such nucleic acid molecules can be obtained in a variety of ways including traditional cloning techniques using oligonucleotide probes of to screen appropriate libraries or DNA and PCR amplification of appropriate libraries or DNA using oligonucleotide primers. Preferred libraries to screen or from which to amplify nucleic acid molecule include bacterial and yeast genomic DNA libraries, and in particular, Escherichia coli genomic DNA libraries. Techniques to clone and amplify genes are disclosed, for example, in Sambrook et al., ibid.

The present invention includes a recombinant vector, which includes at least one isolated nucleic acid molecule of the present invention, inserted into any vector capable of delivering the nucleic acid molecule into a bacterial cell. Such a vector can contain bacterial nucleic acid sequences that are not naturally found adjacent to the isolated nucleic acid molecules to be inserted into the vector. The vector can be either RNA or DNA and typically is a plasmid. Recombinant vectors can be used in the cloning, sequencing, and/or otherwise manipulating of nucleic acid molecules. One type of recombinant vector, referred to herein as a recombinant molecule and described in more detail below, can be used in the expression of nucleic acid molecules. Preferred recombinant vectors are capable of replicating in a transformed bacterial or yeast cell, and in particular, in an Escherichia coli cell.

Transformation of a nucleic acid molecule into a cell can be accomplished by any method by which a nucleic acid molecule can be inserted into the cell. Transformation techniques include, but are not limited to, transfection, electroporation and microinjection.

A recombinant cell is preferably produced by transforming a bacterial cell with one or more recombinant molecules, each comprising one or more nucleic acid molecules operatively linked to an expression vector containing one or more transcription control sequences. The phrase, operatively linked, refers to insertion of a nucleic acid molecule into an expression vector in a manner such that the molecule is able to be expressed when transformed into a host cell. As used herein, an expression vector is a DNA or RNA vector that is capable of transforming a host cell and of effecting expression of a specified nucleic acid molecule. Preferably, the expression vector is also capable of replicating within the host cell. In the present invention, expression vectors are typically plasmids. Expression vectors of the present invention include any vectors that function (i.e., direct gene expression) in a yeast host cell or a bacterial host cell, preferably an Escherichia coli host cell.

Nucleic acid molecules of the present invention can be operatively linked to expression vectors containing regulatory sequences such as transcription control sequences, translation control sequences, origins of replication, and other regulatory sequences that are compatible with the recombinant cell and that control the expression of nucleic acid molecules of the present invention. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in yeast or bacterial cells and preferably, Escherichia coli. A variety of such transcription control sequences are known to those skilled in the art.

It may be appreciated by one skilled in the art that use of recombinant DNA technologies can improve expression of transformed nucleic acid molecules by manipulating, for example, the number of copies of the nucleic acid molecules within a host cell, the efficiency with which those nucleic acid molecules are transcribed, the efficiency with which the resultant transcripts are translated, and the efficiency of post-translational modifications. Recombinant techniques useful for increasing the expression of nucleic acid molecules of the present invention include, but are not limited to, operatively linking nucleic acid molecules to high-copy number plasmids, integration of the nucleic acid molecules into the host cell chromosome, addition of vector stability sequences to plasmids, substitutions or modifications of transcription control signals (e.g., promoters, operators, enhancers), substitutions or modifications of translational control signals, modification of nucleic acid molecules of the present invention to correspond to the codon usage of the host cell, deletion of sequences that destabilize transcripts, and use of control signals that temporally separate recombinant cell growth from recombinant enzyme production during fermentation. The activity of an expressed recombinant protein of the present invention may be improved by fragmenting, modifying, or derivatizing nucleic acid molecules encoding such a protein.

The following experimental results are provided for the purposes of illustration and are not intended to limit the scope of the invention.

### EXAMPLES

### Example 1

The following example describes the production of mutant Escherichia coli strains which are blocked in amino acid sugar metabolic pathways involving degradation of N-glucosamine.

The starting strain for the construction of all N-glucosamine overproducing strains described herein was E. coli W3110 (publicly available from the American Type Culture Collection as ATCC No. 25947), a prototrophic, F⁻ λ⁻ derivative of E. coli K-12 (Bachmann, 1987, “Escherichia coli and Salmonella typhimurium”, Cellular and Molecular Biology, pp.1190-1219; incorporated herein by reference in its entirety). A list of all strains used and produced in the following examples is provided in Table 1.

TABLE 1
Bacterial strains.

| Strain | Alias | Genotype | Source/Reference |
|---|---|---|---|
| W3110 | | F⁻³¹ mcrA mcrB IN(rrnD-rrnE)1 λ⁻ | ATCC |
| IBPC 522 | | thi-1 argG6 argE3 his-4 mtl-1 xyl-5 | J. Plumbridge |
| | | rpsL tsx-29? DlacX74 manXYZ8 | |
| | | nagE47 ptsG22 zcf-229::Tn10 | |
| IBPC 566 | | thi-1 argG6 argE3 his-4 mtl-1 xyl-5 | J. Plumbridge |
| | | rpsL tsx-29? ΔlacX74 manXYZ8 zdj- | |
| | | 225::Tn10 | |
| IBPC 590 | | thi-1 argG6 argE3 his-4 mtl-1 xyl-5 | J. Plumbridge |
| | | rpsL tsx-29? ΔlacX74 Δnag::TcR | |
| 7101-6 | W3110 | F⁻ mcrA mcrB IN(rrnD-rrnE)1 λ⁻ | W3110 × P1ᵥᵢᵣ(IBPC566) |
| | ptsM | manXYZ8 zdj-225:Tn10 | |
| 7101-7 | W3110 | F⁻ mcrA mcrB IN(rrnD-rrnE)1 λ⁻ | W3110 × P1ᵥᵢᵣ(IBPC566) |
| | ptsM | manXYZ8 zdj-225::Tn10 | |
| 7101-9 | W3110 | F⁻ mcrA mcrB IN(rrnD-rrnE)1 λ⁻ | W3110 × P1ᵥᵢᵣ(IBPC590) |
| | Δnag | Δnag::TcR | |
| 7101-13 | W3110 | F⁻ mcrA mcrB IN(rrnD-rrnE)1 λ⁻ | 7101-6 selected on TCS |
| | ptsM TcS | manXYZ8 zdj-225:Tn10? TcS | medium |
| 7101-14 | W3110 | F⁻ mcrA mcrB IN(rrnD-rrnE)1 λ⁻ | 7101-7 selected on TCS |
| | ptsM TcS | manXYZ8 zdj-225::Tn10? TcS | medium |
| 7101-15 | W3110 | F⁻ mcrA mcrB IN(rrnD-rrnE)1 λ⁻ | 7101-14 × P1ᵥᵢᵣ(IBPC522) |
| | ptsM ptsG | manXYZ8zdj-225::Tn10? ptsG22 | |
| | | zcf-229::Tn10 | |
| 7101-17 | W3110 | F⁻ mcrA mcrB IN(rrnD-rrnE)1 λ⁻ | 7101-13 × P1ᵥᵢᵣ(IBPC590) |
| | ptsM Δnag | manXYZ8 zdj-225::Tn10? TcS | |
| | | Δnag::TcR | |
| 7101-22 | W3110 | F⁻ mcrA mcrB IN(rrnD-rrnE)1 λ⁻ | 7101-15 selected on TCS |
| | ptsM ptsG | manXYZ8 zdj-225::Tn10? ptsG22 | medium |
| | TcS | zcf-229::Tn10? TcS | |
| 2123-4 | W3110 | F⁻ mcrA mcrB IN(rrnD-rrnE)1 λ⁻ | 7101-22 × P1ᵥᵢᵣ(IBPC590) |
| | ptsM ptsG | manXYZ8 zdj-225:Tn10? ptsG22 | |
| | Δnag | zcf-229::Tn10? TcS Δnag::TcR | |
| W3110(DE3) | | F⁻ mcrA mcrB IN(rrnD-rrnE)1 | W3110 lysogenized with |
| | | λDE3 | λDE3 |
| 7101-9(DE3) | | F⁻ mcrA mcrB IN(rrnD-rrnE)1 | 7101-9 lysogenized with |
| | | λDE3 Δnag::TcR | λDE3 |
| 7101-17(DE3) | | F⁻ mcrA mcrB | 7101-17 lysogenized |
| | | IN(rrnD-rrnE)1 | with λDE3 |
| | | λDE3 manXYZ8 zdj- | |
| | | 225::Tn10? TcS | |
| | | Δnag::TcR | |
| 2123-4(DE3) | | F⁻ mcrA mcrB | 2123-4 lysogenized |
| | | IN(rrnD-rrnE)1 | with λDE3 |
| | | λDE3 manXYZ8 zdj- | |
| | | 225::Tn10? ptsG22 | |
| | | zcf-229::Tn10 TcS | |
| | | Δnag::TcR | |
| BL21(DE3) | | F⁻ ompT hsdS_{B} gal | Novagen, Inc. |
| | | dcm λDE3 | |
| ATCC 47002 | JC7623 | F⁻ recB21 recC22 | ATCC |
| | | sbcB15 leu-6 ara-14 | |
| | | his-4 λ⁻ | |
| T-71 | | F⁻ recB21 recC22 | Integration of pT7- |
| | | sbcB15 leu-6 ara-14 | glmS-Cm into lacZ of |
| | | his-4 λ⁻ lacZ::pT7- | ATCC47002 by |
| | | glmS-Cm8H7 | transformation with |
| | | | pKLN23-28 |
| T-81 | | F⁻ recB21 reC22 | Integration of pT7- |
| | | sbcB15 leu-6 ara-14 | glmS-Cm into lacZ of |
| | | his-4 λ⁻ lacZ::pT7- | ATCC47002 by |
| | | glmS-Cm8H8 | transformation with |
| | | | pKLN23-28 |
| 2123-5 | | W3110(DE3) | W3110(DE3) × |
| | | lacZ::pT7-glmS- | P1ᵥᵢᵣ(T-71) |
| | | Cm8H7 | |
| 2123-6 | | W3110(DE3) | W3110(DE3) × |
| | | lacZ::pT7-glmS- | P1ᵥᵢᵣ(T-81) |
| | | Cm8H8 | |
| 2123-7 | | W3110(DE3) | W3110(DE3) × |
| | | lacZ::pT7-glmS- | P1ᵥᵢᵣ(T-71) |
| | | Cm8H7 | |
| 2123-8 | | W3110(DE3) | W3110(DE3) × |
| | | lacZ::pT7-glmS- | P1ᵥᵢᵣ(T-81) |
| | | Cm8H8 | |
| 2123-9 | | 7101-9(DE3) | 7101-9(DE3) × |
| | | lacZ::pT7-glmS- | P1ᵥᵢᵣ(T-71) |
| | | Cm8H7 | |
| 2123-10 | | 7101-9(DE3) | 7101-9(DE3) × |
| | | lacZ::pT7-glmS- | P1ᵥᵢᵣ(T-81) |
| | | Cm8H8 | |
| 2123-11 | | 7101-17(DE3) | 7101-17(DE3)× |
| | | lacZ::pT7-glmS- | P1ᵥᵢᵣ(T-71) |
| | | Cm8H7 | |
| 2123-12 | | 7101-17(DE3) | 7101-17(DE3) × |
| | | lacZ::pT7-glmS- | P1ᵥᵢᵣ(T-81) |
| | | Cm8HS | |
| 2123-13 | | 2123-4(DE3) | 2123-4(DE3) × P1(T- |
| | | lacZ::pT7-glmS- | 71) |
| | | Cm8H7 | |
| 2123-14 | | 2123-4(DE3) | 2123-4(DE3) × P1(T- |
| | | lacZ::pT7-glmS- | 81) |
| | | Cm8H8 | |
| NovaBlue | | endA1 hsdR17 | Novagen |
| | | supE44 thi-1 recA1 | |
| | | gyrA96 relA1 lac [F' | |
| | | proA⁺B⁺ | |
| | | lacI^{q}ZΔM15::Tn10] | |
| LE392 | | F⁻ e14⁻ (McrA⁻) | Lab collection |
| | | hsdR514(r⁻m⁺) | |
| | | supE44 supFS8 lacY1 | |
| | | or Δlac(IZY)6 galK2 | |
| | | galT22 metB1 trpR55 | |
| 2123-16 | | LE392 glmS13 | NG mutagenesis of |
| | | | LE392 |
| 2123-49 | | 7101-17(DE3) | Error-prone PCR |
| | | lacZ::pT7-glmS11C- | with pKLN23-28; |
| | | Cm8H8 | integration of mutant |
| | | | glmS into |
| | | | ATCC47002; transfer |
| | | | to 7101-17(DE3) by |
| | | | P1 transduction |
| 2123-51 | | 7101-17(DE3) | Error-prone PCR |
| | | lacZ::pT7-glmS52B- | with pKLN23 -28; |
| | | CmSH8 | integration of mutant |
| | | | gimS into |
| | | | ATCC47002; transfer |
| | | | to 7101-17(DE3) by |
| | | | P1 transduction |
| 2123-54 | | 7101-17(DE3) | Error-prone PCR |
| | | lacZ::pT7-glmS8A- | with pKLN23-28; |
| | | Cm8H8 | integration of mutant |
| | | | glmS into |
| | | | ATCC47002; transfer |
| | | | to 7101-17(DE3) by |
| | | | P1 transduction |

Host strains blocked for N-glucosamine uptake and degradation were constructed by introducing mutations in the nagE, manXYZ and ptsG genes, which block transport of N-glucosamine, and the nagA, -B, -C, and -D genes, which are involved in metabolism of N-glucosamine-6-phosphate. Each of these genes has been described in detail previously herein. Mutations in these genes were introduced into strains using the transducing bacteriophage P1ᵥᵢᵣ (as described in Miller, 1972, “Experiments in Molecular Genetics”, Cold Spring Harbor Laboratory, which is incorporated herein by reference in its entirety).

In this technique, genes or mutations from one strain (the donor strain) are transferred to a recipient strain using the bacteriophage. When bacteriophage P1ᵥᵢᵣ is grown on the donor strain, a small portion of the phage particles that are produced contain chromosomal DNA from the donor rather than the normal complement of phage DNA. Upon infection of the recipient strain with bacteriophage grown on the donor strain, those bacteriophage particles containing chromosomal DNA from the donor strain can transfer that DNA to the recipient strain. If there is a strong selection for the DNA from the donor strain, recipient strains containing the appropriate gene or mutation from the donor strain can be selected.

To grow P1ᵥᵢᵣ on a donor strain, an existing bacteriophage stock was used to infect a culture of that strain. The recipient strain was grown at 37° C. in LBMC medium (10 g/L Bacto tryptone, 5 g/L yeast extract, 10 g/L NaCl, 1 mM MgCl₂, 5 mM CaCl₂) until the absorbance at 600 nm was approximately 1.0, corresponding to approximately 6×10⁸ cells per mL of culture. One mL of the culture was then infected with a dilution of the phage stock at a ratio of approximately one phage per 10 cells. The mixture was incubated without shaking for 20 minutes at 37° C., then transferred to 10 mL of prewarmed LBMC broth in a 125 mL baffled Erlenmeyer flask. The resulting culture was shaken vigorously for 2-3 hours at 37° C. During this period, it was generally observed that the culture would become more turbid, indicating bacterial growth. Toward the end of this incubation period, the culture would become clear, indicating cell lysis due to bacteriophage growth. After lysis had occurred, the culture was cooled on ice, a few drops of chloroform were added, and the flask was shaken briefly. The contents of the flask were then centrifuged to remove the cell debris and chloroform, and the resulting supernatant generally contained between 10⁸ and 10⁹ bacteriophage per mL.

Mutations were transferred to recipient strains by transduction with P1ᵥᵢᵣ grown on the appropriate donor strain as described above. For transduction with P1_{vir,} a culture of the recipient strain was grown overnight at 37° C. in LBMC broth. 0.1 mL of culture was mixed with 0.1 mL of bacteriophage lysate or a serial dilution of the lysate in a sterile test tube and incubated at 37° C. for 20 minutes. 0.2 mL of 1 M sodium citrate was added to the tube, and the mixture was plated to selective medium. For each transduction, controls containing uninfected cells and bacteriophage lysates without cells were performed as described above. For the production of strains blocked in N-glucosamine degradation, selections were for tetracycline resistance as described below. Tetracycline resistant mutants were selected by plating to LB medium (10 g/L Bacto tryptone, 5 g/L yeast extract, 10 g/L NaCl) containing 12.5 μg/mL tetracycline and 10 mM sodium citrate.

The mutations in the nag genes were introduced simultaneously as a deletion mutation (Δnag: :Tc^{R}). In strain IBPC590 (Plumbridge, Table 1), which contains this mutation, the nag genes have been replaced by a tetracycline-resistance (Tc^{R}) determinant. As a result, the mutation which removes the nag functions was transferred to appropriate recipient hosts by selection for tetracycline resistance. In this case, since the Tc^{R} determinant was contained within the mutation of interest, the Δnag and Tc^{R} mutations were 100% linked. That is, all of the recipient strains receiving the Tc^{R} determinant from IBPC590 also received the Δnag mutation. This was confirmed by examining the growth phenotype of the tetracycline resistant strains resulting from infection with P1ᵥᵢᵣ grown on IBPC590. All such strains were unable to grow on media containing N-glucosamine or N-acetylglucosamine as carbon sources, indicating the presence of the Δnag mutation.

Mutations in the manXYZ and ptsG genes were also introduced by P1ᵥᵢᵣ transduction using phage grown on strains IBPC566 and IBPC522 (Plumbridge, Table 1), respectively. These strains also contained tetracycline-resistance determinants linked to the mutations of interest (designated zdj-225::Tn10 and zcf-229::Tn10, respectively). In these strains, the Tc^{R} determinants were not within the gene but were linked to the gene. Accordingly, not all recipient strains receiving the Tc^{R} determinant contained the mutations of interest. The degree of linkage is indicative of the distance on the chromosome between the Tc^{R} determinant and the mutation of interest. As a result, it was necessary to screen tetracycline resistant strains for manXYZ and ptsG. The manXYZ strains grew slowly on mannose and failed to grow on N-glucosamine as sole carbon sources for growth. The ptsG strains grew slowly on glucose as sole carbon source.

Because all of the selections for the mutations described above were for tetracycline resistance, it was necessary to render strains tetracycline sensitive between steps if multiple mutations were to be introduced. To accomplish this, tetracycline-resistant strains were plated to TCS medium (15 g/L agar, 5 g/L Bacto tryptone, 5 g/L yeast extract, 50 mg/L chlortetracycline hydrochloride, 10 g/L NaCl, 10 g/L NaH₂PO₄.H₂O, 12 mg/L fusaric acid, and 0.1 mM ZnCl₂) which selects for tetracycline sensitive mutants (described in Maloy and Nunn, 1981, J. Bacteriol., 145:1110-1112, which is incorporated herein by reference in its entirety). Colonies arising on this medium were purified by restreaking to the same medium, then checking individual colonies for tetracycline sensitivity by plating to LB media with and without 12.5 μg/mL tetracycline.

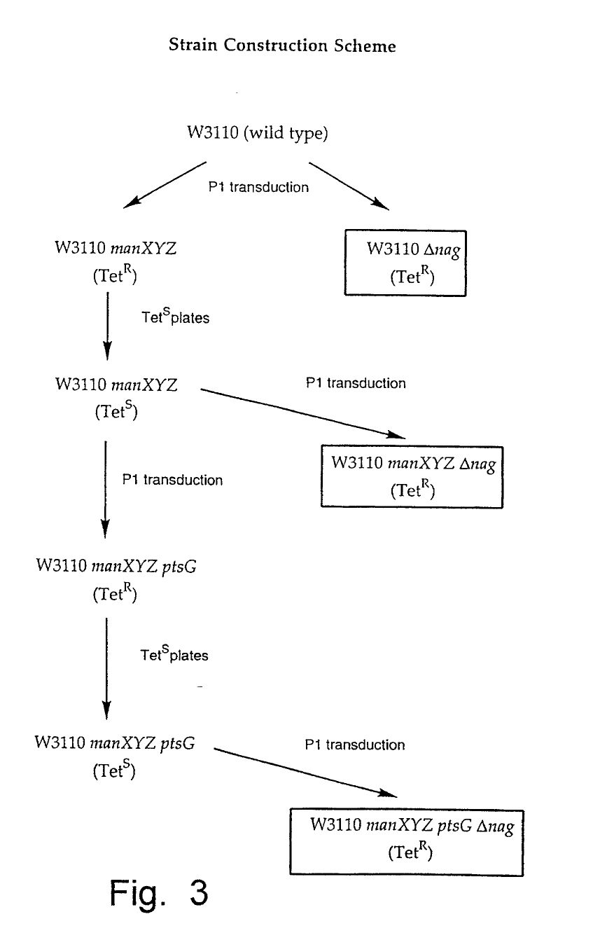

The scheme described above for the production of strains containing combinations of the manXYZ, ptsG, and Δnag mutations is presented schematically in FIG. 3.

### Example 2

The following Example describes the cloning and overexpression of the glmS gene and the integration of the T7-glmS gene cassette into the E. coli chromosome.

### Cloning and Overexpression of the glmS Gene

Using information obtained from the published sequence of the glmS gene (Walker et al., 1984, Biochem. J., 224:799-815, which is incorporated herein by reference in its entirety), primers were synthesized to amplify the gene from genomic DNA isolated from strain W3110 (Table 1) using the polymerase chain reaction (PCR). The primers used for PCR amplification were designated Up1 and Lo8 and had the following sequences:

Up1: 5′-CGGTCTCCCATGTGTGGAATTGTTGGCGC-3′ (SEQ ID NO:1)

Lo8: 5′-CTCTAGAGCGTTGATATTCAGTCAATTACAAACA-3′ (SEQ ID NO:2)

The Up1 primer contained sequences corresponding to the first twenty nucleotides of the glmS gene (represented in nucleotides 10-29 of SEQ ID NO:1) preceded by a BsaI restriction endonuclease site (GGTCT, represented in nucleotides 2-6 of SEQ ID NO:1). The Lo8 primer contained sequences corresponding to positions between 145 and 171 bases downstream of the glmS gene (represented in nucleotides 8-34 of SEQ ID NO:2) preceded by a XbaI restriction endonuclease site (TCTAGA, represented in nucleotides 2-7 of SEQ ID NO:2). PCR amplification was conducted using a standard protocol to generate a fragment of DNA containing the glmS gene with 171 base pairs of DNA downstream of the gene flanked by BsaI and XbaI sites. This DNA fragment was cloned into the vector pCR-Script™SK(+) (Stratagene Cloning Systems, La Jolla, Calif.) using materials and instructions supplied by the manufacturer. The resulting plasmid was designated pKLN23-20.

One consequence of this cloning was that it placed a unique SacI restriction endonuclease site downstream of the gene. This allowed excision of a fragment of DNA containing the glmS gene from pKLN23-20 using the restriction endonucleases BsaI and SacI. This fragment was then cloned between the NcoI and SacI sites of the expression vector pET-24d(+) (Novagen, Inc., Madison, Wis.) to generate plasmid pKLN23-23. The pET-24d(+) expression vector is based on the T7 promoter system (Studier and Moffatt, 1986, J. Mol. Biol., 189:113-130). Cloning in this manner resulted in placement of the glmS gene behind the T7-lac promoter contained on pET-24d(+). The T7-lac promoter is specifically recognized by the T7 RNA polymerase and is only expressed in strains containing a cloned T7 gene 1, which encodes the T7 RNA polymerase. The cloned T7 polymerase gene is contained on a defective bacteriophage λ phage designated λDE3. Strains in which the λDE3 element is integrated into the chromosome contain the T7 RNA polymerase gene driven by the lacUV5 promoter. In those strains, expression of the T7 RNA polymerase gene can be induced using the lactose analog isopropylthio-β-D-galactopyranoside (IPTG) . Accordingly, addition of IPTG to such cultures results in induction of the T7 RNA polymerase gene and expression of any genes controlled by the T7 or T7-lac promoter.

To verify that pKLN23-23 contained the glmS gene driven by the T7-lac promoter, the plasmid was transferred to strain BL21(DE3) (Novagen, Inc.) (Table 1). Strain BL21(DE3)/pKLN23-23 was grown in duplicate in LB medium containing 50 mg/L kanamycin (kanamycin resistance is encoded by the plasmid). One of the duplicates was induced with 1 mM IPTG; the other was not. When the total proteins were examined from these two cultures by sodium dodecyl sulfate polyacrylamide gel electrophoresis, a prominent protein of approximately 70,000 molecular weight, corresponding to the predicted size for the glmS gene product, was observed in cells from the induced culture but not in cells from the uninduced culture. A preliminary enzyme assay from an induced culture indicated several hundred fold higher N-glucosamine-6-phosphate synthase activity in the induced culture than in what had typically been observed in a wild type strain.

### Integration of the T7-glmS Gene Cassette Into the E. coli Chromosome

The glmS gene driven by the T7-lac (the T7-glmS gene cassette) promoter was transferred to the chromosome of E. coli strains by a multistep process. First, the cassette was cloned into plasmid pBRINT-Cm (Balbás et al., 1996, Gene 96:65-69), which is incorporated herein by reference in its entirety) . The gene cassette was then integrated into the chromosome of strain ATCC47002 (Table 1) by the techniques described by Balbás et al., 1996, supra, to generate strains T-71 and T-81 (Table 1). Finally, the gene cassette was transferred to strains of interest by transduction with P1ᵥᵢᵣ, as described below.

The T7-glmS cassette was excised from pKLN23-23 by performing a partial digest of the plasmid with restriction endonuclease BglII and a complete digest with restriction endonuclease HindIII. Plasmid pKLN23-23 contains a BglII site approximately 20 base pairs upstream of the T7 promoter. The glmS gene also contains two BglII sites. A partial digest with this enzyme was necessary to cut the plasmid upstream of the T7 promoter while avoiding the two internal sites. Plasmid pKLN23-23 also contains a unique HindIII site downstream of the glmS gene. The excised T7-glmS cassette was then cloned between the unique BamHI and HindIII sites of pBRINT-Cm. This resulted in the production of plasmids designated pKLN23-27 and pKLN23-28. Plasmids pKLN23-27 and pKLN23-28 contain the T7-glmS cassette in addition to a chloramphenicol resistance determinant flanked by the 5′- and 3′-termini of the E. coli lacZ gene.

Strain ATCC 47002 (Table 1) contains mutations that confer upon it an inability to maintain plasmids such as pBRINT-Cm which contain a ColE1 origin of replication. When such plasmids are transferred to this strain, selection for genetic markers contained on the plasmid results in integration of the plasmid into the chromosome (Balbás et al., 1996, supra) As mentioned above, plasmids pKLN23-27 and -28 contain the T7-glmS cassette and a chloramphenicol resistance determinant flanked by the 5′-and 3′-termini of the E. coli lacZ gene. The lacZ sequences target the incoming DNA to the lacZ gene contained in the chromosome. Integration at the lacZ locus replaces the intact lacZ gene, which encodes the enzyme β-galactosidase, with a partial lacZ gene interrupted by the T7-glmS-Cm cassette. As a result, integration at lacZ results in the strain becoming β-galactosidase negative. The plasmid also contains an ampicillin resistance determinant remote from the 5′-lacZ-T7-glmS-Cm-lacZ-3′ cassette. Integration at lacZ and plasmid loss also results in ampicillin sensitivity.

Plasmid pKLN23-27 and -28 were transferred to strain ATCC 47002, and cells were plated to LB medium containing 10 μg/mL chloramphenicol, 1 mM IPTG, and 40 μg/mL 5-bromo-4-chloro-3-indolyl-βD-galactopyranoside (X-gal). The X-gal contained in the medium is a chromogenic β-galactosidase substrate. Hydrolysis of X-gal by β-galactosidase results in a blue derivative. Inclusion of X-gal and IPTG, which induces the native lacZ gene, results in blue lacZ⁻-positive colonies and white 7lacZ⁻-negative colonies. White (lacZ-negative) chloramphenicol resistant colonies were then selected and purified. The strains were then verified for sensitivity to ampicillin by plating to LB media with and without 100 μg/mL ampicillin. DNA integration was further confirmed using a PCR scheme as described by Balbás et al., 1996, supra. Integrants T-71 and T-81 (Table 1) resulted from the integration of the desired segments of plasmids pKLN23-27 and pKLN23-28, respectively, into the chromosome of ATCC 47002.

The T7-glmS-Cm cassette was then transferred to strains W3110(DE3), 7101-9(DE3), 7101-17(DE3), and 2123-4(DE3) by P1ᵥᵢᵣ transduction, as described in Example 1, using lysates prepared on strains T-71 and T-81. These strains contain various combinations of the Δnag, manXYZ, and ptsG mutations in addition to the λDE3 element necessary for expression from the T7-lac promoter. The λDE3 element was introduced to these strains using the λDE3 lysogenization kit produced by Novagen, Inc. (Madison, Wis.). Transductants were selected on LB agar plates containing 30 μg/mL chloramphenicol and 10 mM sodium citrate. Loss of μ-galactosidase activity was verified on plates containing X-gal and IPTG and DNA integration was further confirmed using a PCR scheme as described by Balbás et al., 1996, supra.

N-glucosamine-6-phosphate synthase activity was measured in production strains containing integrated T7-glmS cassettes after growth in LB medium with and without IPTG (Table 2). N-glucosamine-6-phosphate synthase was assayed in crude cell extracts using either calorimetric or spectrophotometric assays (Badet et al., 1987, Biochemistry 26:1940-1948) as described below. The extracts used for those assays were prepared by suspending washed cell pellets in 5 mL of 0.1 M KH₂PO₄/K₂HPO₄, pH 7.5 per gram of wet cell paste, passing the suspension through a French press at 16,000 psi, and centrifuging the disrupted cell suspension at 35,000-40,000 ×g for 15 to 20 minutes. The supernatant was used as the source of enzyme for the assay.

For a calorimetric assay, 1 mL reactions were prepared containing 45 mM KH₂PO₄/K₂HPO₄, 20 mM fructose-6-phosphate, 15 mM L-glutamine, 2.5 mM EDTA, pH 7.5, and cell extract. The reactions were incubated at 37° C. for 20 minutes and stopped by boiling for 4 minutes. The resulting precipitate was removed by centrifugation and the supernatant was assayed for N-glucosamine-6-phosphate by a modification of the method of Elson and Morgan (1933, Biochem. J. 27:1824-1828) essentially as described by Zalkin (1985, Meth. Enzymol. 113:278-281), both publications of which are incorporated herein by reference in their entireties. To 100 μL of the above supernatant was added 12.5 μL of saturated NaHCO₃ and 12.5 μL of cold, freshly prepared 5% aqueous acetic anhydride. After incubating for 3 minutes at room temperature, the mixture was boiled for 3 minutes to drive off excess acetic anhydride. After cooling to room temperature, 150 μL of 0.8 M potassium borate, pH 9.2 (0.8 M H₃BO₃ adjusted to pH 9.2 with KOH) was added and the mixture was boiled for 3 minutes. After cooling to room temperature, 1.25 mL Ehrlich's reagent (1% p-dimethylaminobenzaldehyde in glacial acetic acid containing 0.125 N HCl) was added to each tube. After incubating at 37° C. for 30 minutes, the absorbance at 585 nm was measured and the amount of N-glucosamine-6-phosphate formed was determined using a standard curve. In the absence of the substrate, fructose-6-phosphate, or when boiled extract was used in the assay, no significant absorbance at 585 nm was observed.

In the spectrophotometric assay, 1 mL reactions containing 50 mM KH₂PO₄/K₂HPO₄, 10 mM fructose-6-phosphate, 6 mM L-glutamine, 10 mM KCl, 0.6 mM acetylpyridine adenine dinucleotide (APAD), and 50-60 Units of L-glutamic dehydrogenase (Sigma Type II from bovine liver) were run at room temperature. The activity was followed by monitoring the absorbance at 365 nm after the addition of extract and corrected for the small absorbance increase observed in the absence of extract. The activity was calculated using a molar extinction coefficient for APAD of 9100.

TABLE 2
Glucosamine 6-Phosphate Synthase Activity in Production Strains
Containing Integrated T7-glmS Cassettes

| | | Activity, (mmole per minute | |
|---|---|---|---|
| | | per mL of extract) | |
| Strain | Host Genotype | −IPTG | +IPTG |
| 2123-5 | DE3 | 23 | 64 |
| 2123-6 | DE3 | 4 | 4 |
| 2123-7 | DE3 | 23 | 96 |
| 2123-8 | DE3 | 25 | 89 |
| 2123-9 | DE3 Δnag | 26 | 58 |
| 2123-10 | DE3 Δnag | 33 | 67 |
| 2123-11 | DE3 Δnag manXYZ | 32 | 59 |
| 2123-12 | DE3 Δnag manXYZ | 17 | 67 |
| 2123-13 | DE3 Δnag manXYZ ptsG | 21 | 68 |
| 2123-14 | DE3 Δnag manXYZ ptsG | 20 | 88 |

Table 2 shows that, on average, the activity of N-glucosamine-6-phosphate synthase in production strains containing integrated T7-glmS cassettes was about three-to four-fold higher with IPTG induction than without. The activities were substantially higher than those obtained with a wild type glmS strain driven by its native promoter, which typically were in the range of 0.05-0.1 μmole per minute per mL of extract. One of the strains, 2123-6, apparently suffered an aberrant integration event since the activity was lower than that observed in the other strains and was not influenced by the presence of IPTG in the medium.

### EXAMPLE 3

The following example shows the effect of strain genotype on N-glucosamine accumulation.

Strains with T7-glmS integrants, produced as described in Example 2, as well as the corresponding parent strains without integrated DNA, were grown in shake flasks containing M9A medium (14 g/L K₂HPO₄, 16 g/L KH₂PO₄, 1 g/L Na₃Citrate.2H₂O, 5 g/L (NH₄)₂SO_{4,} pH 7.0) supplemented with 20 g/L glucose, 10 mM MgSO₄, 1 mM CaCl₂, and 1 mM IPTG. Samples were taken periodically over the course of two days, and the N-glucosamine concentration in the culture supernatant was measured using the modified Elson-Morgan assay as described in Example 2. Samples were assayed with and without acetic anhydride treatment, and the amount of N-glucosamine present was determined from the net absorbance using a standard curve.

Glucosamine concentrations after 24 hours of cultivation, at which time the concentration peaked, are indicated in Table 3. The results shown in Table 3 indicate that for significant N-glucosamine production to occur, the T7-glmS gene cassette must be present. The data also indicate that the presence of the Δnag mutation in the host results in a significant increase in N-glucosamine accumulation compared with its absence. Little effect of the manXYZ mutation was observed in this experiment. In further shake flask experiments, however, strain 2123-12 accumulated slightly higher N-glucosamine concentrations on a consistent basis.

TABLE 3
Glucosamine in Culture Supernatants of Production Strains

| | | Glucosamine |
|---|---|---|
| | | Concentration, |
| Strain | Genotype | mg/L (24 hours) |
| 2123-5 | DE3, T-71 integrant | 21 |
| 2123-7 | DE3, T-71 integrant | 23 |
| 2123-9 | DE3 Δnag, T-71 integrant | 67 |
| 2123-10 | DE3 Δnag, T-81 integrant | 80 |
| 2123-11 | DE3 Δnag manXYZ, T-71 integrant | 65 |
| 2123-12 | DE3 Δnag manXYZ, T-81 integrant | 63 |
| W3110(1DE3) | DE3, no integrant | 4 |
| 7101-9(1DE3) | DE3 Δnag, no integrant | 0 |
| 7101-17(1DE3) | DE3 Δnag manXYZ, no integrant | 0 |

### Example 4

The following example demonstrates the effect feeding nutrients to the cultures has on N-glucosamine accumulation.

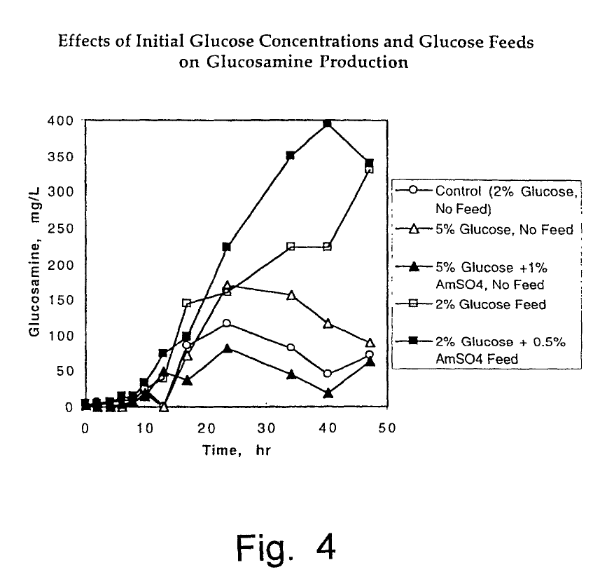

In early experiments, it was observed that N-glucaosamine accumulation ceased when glucose was depleted from cultures. In the experiment summarized by Table 4 and FIG. 4, it was found that increased N-glucosamine accumulation could be accomplished by feeding additional glucose and ammonium sulfate as they became depleted. For this experiment, strain 2123-12 was grown in M9A medium supplemented with 10 mM MgSO₄, 1 mM CaCl₂, and 1 mM IPTG. Initial glucose concentrations and feeding regimens were varied as indicated in Table 4. In one of the flasks, the initial ammonium sulfate concentration was 10 g/L rather than the 5 g/L normally used in M9A medium. Glucose concentration was monitored in shake flasks during cultivation using Diastix® glucose test strips (Bayer Corporation Diagnostics Division, Elkhart, Ind.). When the glucose concentration was at or near depletion (<5 g/L remaining), glucose and/or ammonium sulfate were supplemented as indicated in Table 4. pH was also monitored during cultivation. When the pH varied significantly from the initial pH of 7.0, it was adjusted to 7.0 with sodium hydroxide.

TABLE 4
Shake Flask Experiment to Examine the Effect of Glucose Feeding

| | | Initial | |
|---|---|---|---|
| Flask | Initial | Ammonium | |
| No. | Glucose, g/L | Sulfate, g/L | Feed |
| 1 | 20 | 5 | None |
| 2 | 50 | 5 | None |
| 3 | 50 | 10 | None |
| 4 | 20 | 5 | 20 g/L Glucose |
| 5 | 20 | 5 | 20 g/LGlucose + 5 g/L AmSO₄ |

As FIG. 4 indicates, increasing the supply of glucose had a positive effect on N-glucosamine accumulation. By periodically feeding with glucose and ammonium sulfate (20 g/L and 5 g/L additions, respectively), a maximum accumulation of 0.4 g/L of N-glucosamine was observed, approximately four-fold higher than what was observed in the absence of feeding.

### Example 5

The following example describes the isolation of mutant glmS genes encoding N-glucosamine-6-phosphate synthase enzymes with decreased sensitivity to N-glucosamine-6-phosphate product inhibition.

White (1968, Biochem. J., 106:847-858) first demonstrated that N-glucosamine-6-phosphate synthase was inhibited by N-glucosamine-6-phosphate. Using the spectrophotometric assay for N-glucosamine-6-phosphate synthase as described in Example 2, the effects of N-glucosamine-6-phosphate and N-glucosamine on N-glucosamine-6-phosphate synthase were measured. For determination of product inhibition, assays were run in the presence of various concentrations of added N-glucosamine-6-phosphate.

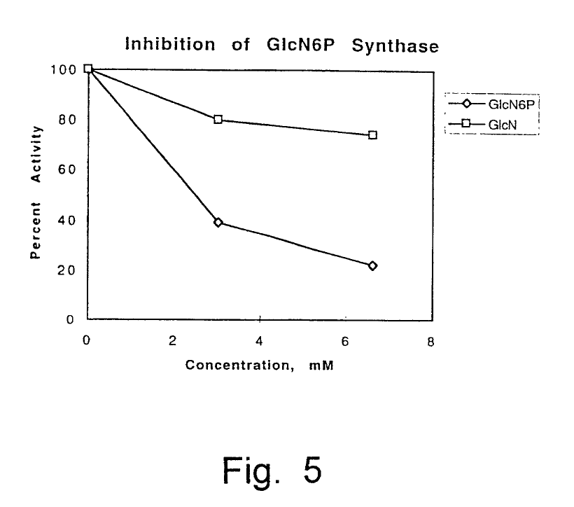

As indicated in FIG. 5, the enzyme is significantly inhibited by N-glucosamine-6-phosphate and slightly inhibited by N-glucosamine. These results are similar to those obtained by White, 1968, supra. This inhibition may be a key factor in limiting N-glucosamine accumulation in the N-glucosamine production strains.

To further increase N-glucosamine synthesis in production strains, efforts were made to isolate mutants of the glmS gene encoding N-glucosamine-6-phosphate synthase variants with reduced product inhibition. To accomplish this, the cloned gene was amplified using the technique of error-prone PCR. In this method, the gene is amplified under conditions that lead to a high frequency of misincorporation errors by the DNA polymerase used for the amplification. As a result, a high frequency of mutations are obtained in the PCR products.

Plasmid pKLN23-28 contains a unique SpeI restriction endonuclease site 25 base pairs upstream of the T7 promoter and 111 base pairs upstream of the start of the glmS gene. The plasmid also contains a unique HindIII site 177 base pairs downstream of the glmS gene. PCR primers of the following sequences were synthesized to correspond to regions just upstream of the SpeI and downstream of the HindIII sites, respectively:

5′-ATGGATGAGCAGACGATGGT-3′ (SEQ ID NO:3)

5′-CCTCGAGGTCGACGGTATC-3′ (SEQ ID NO:4)

Amplification with these primers (SEQ ID NO:3 and SEQ ID NO:4) allowed mutagenesis of a 2119 base pair region that included the entire glmS gene. PCR conditions were as described by Moore and Arnold, 1996, Nature Biotechnology 14:458-467, which is incorporated herein by reference in its entirety. Briefly, a 100 μL solution was prepared containing 1 mM each of the four deoxynucleotide triphosphates, 16.6 mM ammonium sulfate, 67 mM Tris-HCl, pH 8.8, 6.1 mM MgCl₂, 6.7 μM EDTA, 10 mM β-mercaptoethanol, 10 μL DMSO, 30 ng each of the primers (SEQ ID NO:3 and SEQ ID NO:4), either 7 or 35 ng of plasmid pKLN23-28 linearized with Kpn I, and 2.5 Units of Taq DNA polymerase (Perkin Elmer-Cetus, Emeryville, Calif.). The reaction mixture was covered with 70 μL of mineral oil and placed in a thermocycler, where the following steps were repeated for 25 cycles:

1 minute at 94° C.

1 minute at 42° C.

2 minutes at 72° C.

Under these conditions, an error frequency of approximately one mutation per 1000 base pairs has been reported (Moore and Arnold, 1996, supra). The product of the reaction was recovered, purified, and digested with SpeI and HindIII, and cloned into the SpeI-HindIII backbone fragment of pKLN23-28, which effectively substitutes for the wild type glmS gene on the SpeI-HindIII fragment of pKLN23-28. The cloned DNA was used to transform strain NovaBlue (Novagen, Inc., Madison, Wis.), and the transformed cells were plated to LB agar containing ampicillin. A total of 9000 plasmid-containing colonies were pooled from the ampicillin plates and plasmid DNA was prepared from the pooled cells to generate a library of pKLN23-28 derivative plasmids containing mutations in the cloned glmS gene.

The mutant plasmids generated by error-prone PCR were screened for their ability to confer increased N-glucosamine production in a Δnag manXYZ DE3 host background. This screen was in the form of a bioassay in which the ability of plasmid-containing strains to crossfeed N-glucosamine-requiring strains of E. coli was assessed.

To isolate a N-glucosamine-requiring E. coli strain, strains of E. coli (Sarvas, 1971, J. Bacteriol. 105:467-471; Wu and Wu, 1971, J. Bacteriol. 105:455-466) and Bacillus subtilis (Freese et al., 1970, J. Bacteriol. 101:1046-1062) defective for N-glucosamine-6-phosphate synthase require N-glucosamine or N-acetylglucosamine for growth. An N-glucosamine-requiring strain of E. coli was isolated after mutagenesis with N-methyl-N′-nitro-N-nitrosoguanidine (NG). Strain LE392 (Table 1) was grown in LB medium to a cell density of 6×10⁸ cells per mL. 50 μL of 2.5 mg/mL NG dissolved in methanol was added to 2 mL of this culture and the mixture was incubated at 37° C. for 20 minutes. This treatment resulted in about 10% survival of the strain. The mutagenized cells were harvested by centrifugation, and the cells were washed twice by suspension in 0.9% NaCl and recentrifugation. The washed cells were diluted and plated to nutrient agar medium (NA; 5 g/L Bacto peptone, 3 g/L beef extract, 15 g/L agar) containing 0.2 g/L N-acetylglucosamine at a density of between 50 and 200 colony forming units per plate. Approximately 13,000 colonies were plated. These colonies were replica-plated to NA agar with and without 0.2 g/L N-acetylglucosamine. Twenty-two colonies grew on NA with 0.2 g/L N-acetylglucosamine but not on NA without 0.2 g/L N-acetylglucosamine. These colonies were purified by streaking to NA with 0.2 g/L N-acetylglucosamine, and their growth phenotype was rechecked. Of the original 22 colonies selected, five had the phenotype expected of a glmS mutant of LE392. They failed to grow on NA but grew on NA supplemented with 0.2 g/L of N-glucosamine or 0.2 g/L N-acetylglucosamine. They also failed to grow on glucose minimal agar, but grew on glucose minimal agar supplemented with 0.2 g/L N-acetylglucosamine. One of these mutants was designated 2123-16 (Table 1).

For the cross-feeding assay, agar plates containing either glycerol or fructose as the principle carbon source for growth were overlaid with cells from a culture of strain 2123-16, the N-glucosamine-requiring strain isolated as described above. N-glucosamine-producing strains were stabbed into the agar and the ability to produce N-glucosamine was assessed based on the size of the “halo” of growth of the indicator strain surrounding the stab. Those stabs surrounded by larger halos were considered to produce greater amounts of N-glucosamine.

The media used for the cross-feeding assays consisted of M9 minimal medium (6 g/L Na₂HPO₄, 3 g/L KH₂PO₄, 0.5 g/L NaCl, 1 g/L NH₄Cl, 1 mM MgSO₄, 0.1 mM CaCl₂) supplemented with 40 mg/L of L-methionine (required for growth of strains LE392 or 2123-16) and 2 g/L of either glycerol or fructose. These plates were overlaid with strain 2123-16 as follows. A culture of strain 2123-16 was grown overnight at 37° C. in LB medium containing 1 g/L N-acetylglucosamine. The culture was harvested by centrifugation, and the cells were washed twice by suspension in 0.9% NaCl and recentrifugation. The washed cells were suspended in the original volume of 0.9% NaCl. For each plate to be overlaid, 0.1 mL of washed cell suspension was mixed with 3 mL of molten (50° C.) F-top agar (8 g/L NaCl, 8 g/L agar) and poured onto the plate.

The library of pKLN23-28 mutant plasmids was transferred to strain 7101-17(DE3) and transformed cells were plated to LB agar containing 100 μg/mL ampicillin. Each colony arising on these plates contained an individual member of the mutant plasmid library. The colonies were screened by picking them from the LB+ampicillin plates and stabbing them sequentially into:

(1) LB agar+ampicillin;

(2) glycerol minimal agar overlaid with strain 2123-16; and,

(3) fructose minimal agar overlaid with strain 2123-16

All plates were incubated for about 24 hours at 37° C. After this incubation period, halos of growth of the 2123-16 indicator strain could be observed surrounding the stabs in the overlaid plates. Those colonies giving rise to the larger halos were picked from the corresponding LB+ampicillin plate and streaked for purification. In an initial screen, 4368 mutant candidates were screened, and 96 initial candidates were identified. Upon rescreening those, 30 appeared to be superior to the rest, i.e. resulted in larger halos of the indicator strain.

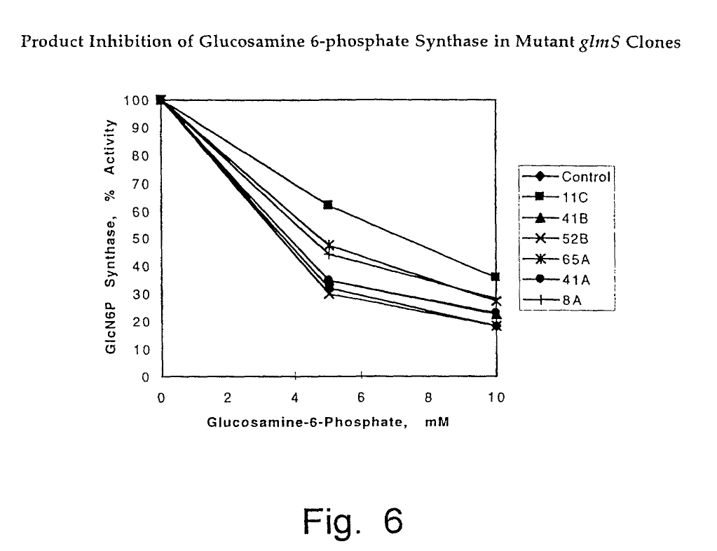

Enzyme assays performed with six of the plasmid-containing strains isolated as described above indicated that three of the strains were less sensitive to inhibition by N-glucosamine-6-phosphate than the enzyme from the control strain 7101-17(DE3)/PKLN23-28. The strains were grown overnight in LB broth containing 100 μg/mL ampicillin and 1 mM IPTG. Extracts prepared from cells harvested from those cultures were assayed for N-glucosamine-6-phosphate synthase using the spectrophotometric assay (described in Example 2) in the presence and absence of added N-glucosamine-6-phosphate. The mutant clones designated 11C, 65A, and 8A were significantly less sensitive to N-glucosamine-6-phosphate than the control strain (FIG. 6). Other mutants were not distinguishable from the control by this assay.

### Example 6

The following example describes the construction and characterization of N-glucosamine production strains with mutations in glmS which result in reduced product inhibition.

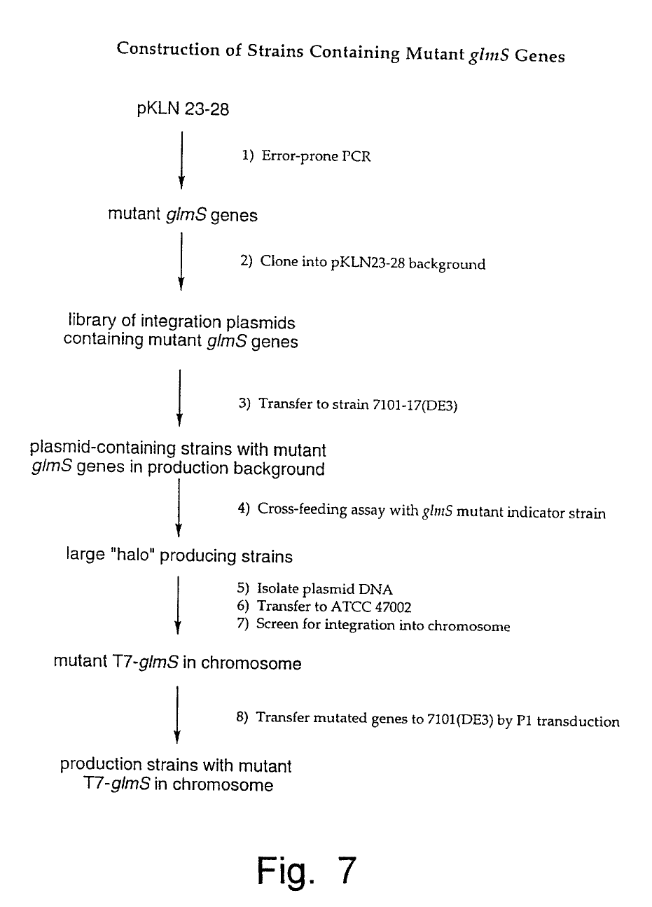

Plasmid DNA isolated from clones 11C, 52B, and 8A described above were transferred to strain ATCC 47002, which had been used previously to integrate the cloned T7-glmS construct into the E. coli chromosome. Integration was readily accomplished using the methods described in Example 2, and the integrated DNA was transferred to strain 7101-17(DE3) by P1 transduction as described in Example 1. These procedures produced strains that have the same genotype as strain 2123-12 except for the presence of mutations in the glmS gene generated by PCR. These new mutant production strains were designated 2123-49, 2323-51, and 2123-54, respectively. A summary of the strain construction strategy is presented in FIG. 7.

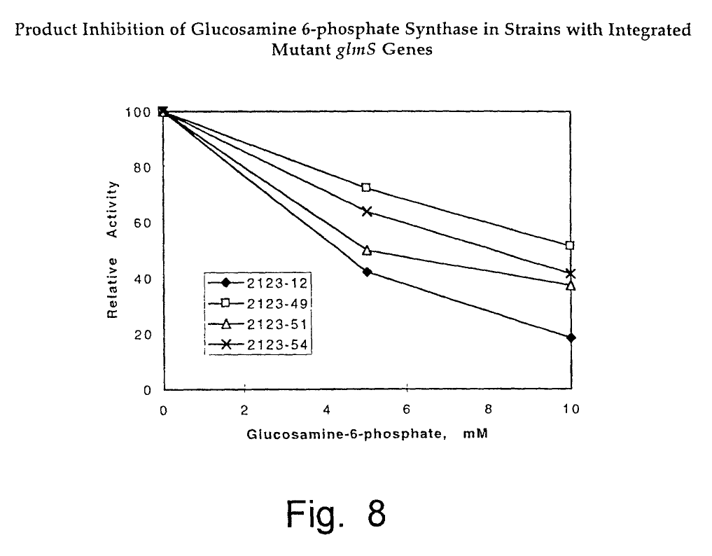

Strains 2123-12, 2123-49, 2123-51, and 2123-54 were grown overnight in LB broth containing 100 μg/mL ampicillin and 1 mM IPTG. Extracts prepared from cells harvested from those cultures were assayed for N-glucosamine-6-phosphate synthase using the spectrophotometric assay described in Example 2 in the presence and absence of added N-glucosamine-6-phosphate. The results of this assay are shown in FIG. 8.

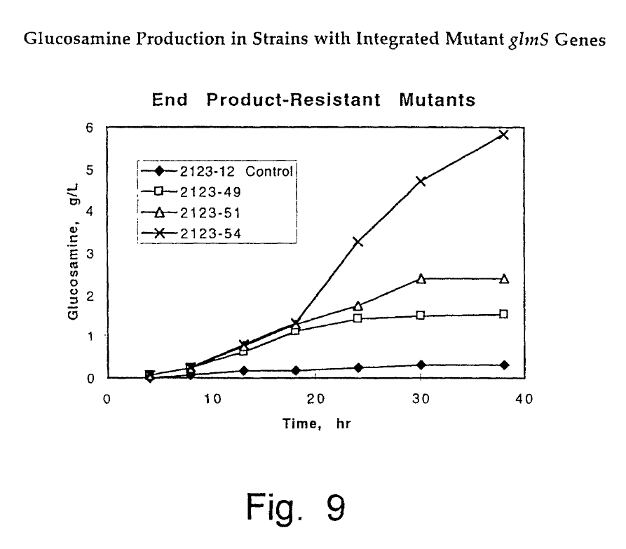

Glucosamine production in these mutants was significantly elevated compared to that in 2123-12. When N-glucosamine production was assayed in shake flask cultures grown using the glucose and ammonium sulfate feeding protocol previously described in Example 4, when the cultures were grown to a cell density of about O.D.⁶⁰⁰14 (about 8.4 g/L by dry cell weight), strains 2123-49, 2123-51, and 2123-54 produced 1.5, 2.4, and 5.8 g/L N-glucosamine, respectively (FIG. 9) compared with 0.3 g/L for 2123-12.

In summary, the present inventors have described herein the use of metabolic engineering to create the first N-glucosamine overproducing strain of E. coli. The concept, proven here, will be generally applicable to any microorganism having a pathway for the production of amino sugars, or to any recombinant microorganism into which a pathway for the production of amino sugars has been introduced. In addition to the present strategy for creating a N-glucosamine-producing strain (i.e., eliminating N-glucosamine degradation and uptake and increasing expression of the glmS gene), the present inventors have also established that reducing product inhibition of N-glucosamine-6-phosphate synthase by N-glucosamine-6-phosphate improves N-glucosamine production.

What is claimed:

1. A method to produce N-glucosamine by fermentation, comprising:
(a) culturing in a fermentation medium comprising assimilable sources of carbon, nitrogen and phosphate, a microorganism having a genetic modification in an amino sugar metabolic pathway, said amino sugar metabolic pathway selected from the group consisting of a pathway for converting N-glucosamine-6-phosphate into another compound, a pathway for synthesizing N-glucosamine-6-phosphate, a pathway for transport of N-glucosamine or N-glucosamine-6-phosphate out of said microorganism, a pathway for transport of N-glucosamine into said microorganism, and a pathway which competes for substrates involved in the production of N-glucosamine-6-phosphate;
wherein said step of culturing produces a product selected from the group consisting of N-glucosamine-6-phosphate and N-glucosamine from said microorganism; and
(b) recovering said product.

2. The method of claim 1, wherein said N-glucosamine-6-phosphate is intracellular and said N-glucosamine is extracellular, wherein said step of recovering comprises a recovering step selected from the group consisting of recovering said N-glucosamine-6-phosphate from said microorganism, recovering said N-glucosamine from said fermentation medium, and a combination thereof.

3. The method of claim 1, wherein said product is N-glucosamine which is secreted into said fermentation medium by said microorganism and wherein said step of recovering comprises purification of said N-glucosamine from said fermentation medium.

4. The method of claim 1, wherein said product is intracellular N-glucosamine-6-phosphate and said step of recovering comprises isolating said N-glucosamine-6-phosphate from said microorganism.

5. The method of claim 1, wherein said product is intracellular N-glucosamine-6-phosphate and said step of recovering further comprises dephosphorylating said N-glucosamine-6-phosphate to produce N-glucosamine.

6. The method of claim 1, wherein said step of culturing comprises maintaining said source of carbon at a concentration of from about 0.5% to about 5% in said fermentation medium.

7. The method of claim 1, wherein said genetic modification is in a gene encoding a protein selected from the group consisting of N-acetylglucosamine-6-phosphate deacetylase, N-glucosamine-6-phosphate deaminase, N-acetyl-glucosamine-specific enzyme II^{Nag}, N-glucosamine-6-phosphate synthase, phosphoglucosamine mutase, N-glucosamine-1-phosphate acetyltransferase-N-acetylglucosamine-1-phosphate uridyltransferase, phosphofructokinase, Enzyme II^{Glc} of the PEP:glucose PTS, EIIM, P/III^{Man} of the PEP:mannose PTS, and alkaline phosphatase.

8. The method of claim 1, wherein said genetic modification comprises transformation of said microorganism with a recombinant nucleic acid molecule encoding N-glucosamine-6-phosphate synthase to increase expression of said N-glucosamine-6-phosphate synthase by said microorganism, wherein said recombinant nucleic acid molecule is operatively linked to a transcription control sequence.

9. The method of claim 8, wherein said recombinant nucleic acid molecule is integrated into the genome of said microorganism.

10. The method of claim 8, wherein said recombinant nucleic acid molecule encoding N-glucosamine-6-phosphate synthase comprises a genetic modification which reduces N-glucosamine-6-phosphate product inhibition of said N-glucosamine-6-phosphate synthase.

11. The method of claim 8, wherein said microorganism has at least one additional genetic modification in a gene encoding a protein selected from the group consisting of N-acetylglucosamine-6-phosphate deacetylase, N-glucosamine-6-phosphate deaminase, N-acetyl-glucosamine-specific enzyme II^{Nag}, phosphoglucosamine mutase, N-glucosamine-1-phosphate acetyltransferase-N-acetylglucosamine-1-phosphate uridyltransferase, phosphofructokinase, Enzyme II^{Glc} of the PEP:glucose PTS, EIIM, P/III^{Man} of the PEP:mannose PTS, and alkaline phosphatase, wherein said genetic modification decreases enzymatic activity of said protein.

12. The method of claim 8, wherein said microorganism has a modification in genes encoding N-acetylglucosamine-6-phosphate deacetylase, N-glucosamine-6-phosphate deaminase and N-acetyl-glucosamine-specific enzyme II^{Nag}, wherein said genetic modification decreases enzymatic activity of said protein.

13. The method of claim 12, wherein said genetic modification is a deletion of at least a portion of said genes.

14. The method of claim 1, wherein said microorganism is selected from the group consisting of bacteria and yeast.

15. The method of claim 1, wherein said microorganism is a bacterium of the genus Escherichia.

16. The method of claim 1, wherein said microorganism is Escherichia coli.

17. The method of claim 16, wherein said genetic modification is a mutation in an Escherichia coli gene selected from the group consisting of nagA, nagB, nagC, nagD, nagE, manXYZ, glmM, pfkB, pfkA, glmU, glmS, ptsG and alkaline phosphatase gene.

18. A method to produce N-glucosamine by fermentation, comprising:
(a) culturing in a fermentation medium comprising assimilable sources of carbon, nitrogen and phosphate, an Escherichia coli transformed with a recombinant nucleic acid molecule encoding N-glucosamine-6-phosphate synthase, wherein said recombinant nucleic acid molecule increases expression of said N-glucosamine-6-phosphate synthase by said Escherichia coli, and wherein said recombinant nucleic acid molecule is operatively linked to a transcription control sequence;
wherein said step of culturing produces a product selected from the group consisting of N-glucosamine-6-phosphate and N-glucosamine from said Escherichia coli; and
(b) recovering said product.

19. The method of claim 18, wherein said recombinant nucleic acid molecule comprises a genetic modification which reduces N-glucosamine-6-phosphate product inhibition of said N-glucosamine-6-phosphate synthase.

20. The method of claim 18, wherein said Escherichia coli has an additional genetic modification in at least one gene selected from the group consisting of nagA, nagB, nagC, nagD, nagE, manXYZ, glmM, pfkB, pfkA, glmU, glmS, ptsG and alkaline phosphatase gene.

21. The method of claim 18, wherein said N-glucosamine-6-phosphate is intracellular and said N-glucosamine is extracellular, wherein said step of recovering comprises a recovering step selected from the group consisting of recovering said N-glucosamine-6-phosphate from said microorganism, recovering said N-glucosamine from said fermentation medium, and a combination thereof.

22. A microorganism for producing N-glucosamine by a biosynthetic process, said microorganism being transformed with a recombinant nucleic acid molecule encoding N-glucosamine-6-phosphate synthase, said recombinant nucleic acid molecule being operatively linked to a transcription control sequence and comprising a genetic modification which reduces N-glucosamine-6-phosphate product inhibition of said N-glucosamine-6-phosphate synthase;
wherein expression of said recombinant nucleic acid molecule increases expression of said N-glucosamine-6-phosphate synthase by said microorganism.

23. The microorganism of claim 22, wherein said recombinant nucleic acid molecule is integrated into the genome of said microorganism.

24. The microorganism of claim 22, wherein said microorganism has at least one additional genetic modification in a gene encoding a protein selected from the group consisting of N-acetylglucosamine-6-phosphate deacetylase, N-glucosamine-6-phosphate deaminase, N-acetyl-glucosamine-specific enzyme II^{Nag}, phosphoglucosamine mutase, N-glucosamine-l-phosphate acetyltransferase-N-acetylglucosamine-1-phosphate uridyltransferase, phosphofructokinase, Enzyme II^{Glc} of the PEP:glucose PTS, EIIM, P/III^{Man} of the PEP:mannose PTS, and alkaline phosphatase, wherein said genetic modification decreases enzymatic activity of said protein.

25. The microorganism of claim 22, wherein said microorganism has a modification in genes encoding N-acetylglucosamine-6-phosphate deacetylase, N-glucosamine-6-phosphate deaminase and N-acetyl-glucosamine-specific enzyme II^{Nag}, wherein said genetic modification decreases enzymatic activity of said protein.

26. The microorganism of claim 25, wherein said genetic modification is a deletion of at least a portion of said genes.

27. The microorganism of claim 22, wherein said microorganism is selected from the group consisting of a yeast and a bacterium.

28. The microorganism of claim 22, wherein said microorganism is a bacterium of the genus Escherichia.

29. The microorganism of claim 22, wherein said microorganism is Escherichia coli.

30. The microorganism of claim 29, wherein said Escherichia coli has at least one additional genetic modification in a gene selected from the group consisting of nagA, nagB, nagC, nagD, nagE, manXYZ, glmM, pfkB, pfkA, glmU, ptsG and alkaline phosphatase gene, wherein said genetic modification decreases enzymatic activity of a protein encoded by said gene.

31. The microorganism of claim 29, wherein said Escherichia coli has a deletion of nag regulon genes.

32. The microorganism of claim 29, wherein said Escherichia coli has a deletion of nag regulon genes and a genetic modification in manXYZ genes such that the proteins encoded by said manXYZ genes have decreased enzymatic activity.

33. The microorganism of claim 22, wherein said microorganism produces at least about 1 g/L of N-glucosamine when cultured for about 24 hours at 37° C. to a cell density of at least about 8 g/L by dry cell weight, in a pH 7.0 fermentation medium comprising: 14 g/L K₂HPO₄, 16 g/L KH₂PO₄, 1 g/L Na₃Citrate.2H₂O, 5 g/L (NH₄)₂SO₄, 20 g/L glucose, 10 mM MgSO₄, 1 mM CaCl₂, and 1 mM IPTG.

34. A microorganism for producing N-glucosamine by a biosynthetic process, said microorganism comprising:
(a) a recombinant nucleic acid molecule encoding N-glucosamine-6-phosphate synthase, said recombinant nucleic acid molecule being operatively linked to a transcription control sequence, wherein expression of said recombinant nucleic acid molecule increases expression of said N-glucosamine-6-phosphate synthase by said microorganism; and,
(b) at least one genetic modification in a gene encoding a protein selected from the group consisting of N-acetylglucosamine-6-phosphate deacetylase, N-glucosamine-6-phosphate deaminase, N-acetyl-glucosamine-specific enzyme II^{Nag}, phosphoglucosamine mutase, N-glucosamine-1-phosphate acetyltransferase-N-acetylglucosamine-1-phosphate uridyltransferase, phosphofructokinase, Enzyme II^{Glc} of the PEP:glucose PTS, EIIM, P/III^{Man} of the PEP:mannose PTS, and alkaline phosphatase, wherein said genetic modification decreases enzymatic activity of said protein.

35. The microorganism of claim 34, wherein said recombinant nucleic acid molecule is integrated into the genome of said microorganism.

36. The microorganism of claim 34, wherein said microorganism is selected from the group consisting of a yeast and a bacterium.

37. The microorganism of claim 34, wherein said microorganism is a bacterium of the genus Escherichia.

38. The microorganism of claim 34, wherein said microorganism is Escherichia coli.

39. The microorganism of claim 34, wherein said microorganism produces at least about 1 g/L of N-glucosamine when cultured for about 24 hours at 37° C. to a cell density of at least about 8 g/L by dry cell weight, in a pH 7.0 fermentation medium comprising: 14 g/L K₂HPO₄, 16 g/L KH₂PO₄, 1 g/L Na₃Citrate.2H₂O, 5 g/L (NH₄)₂SO₄, 20 g/L glucose, 10 mM MgSO₄, 1 mM CaCl₂, and 1 mM IPTG.


SEQUENCE LISTING (4 sequences)

SEQ ID NO: 1 - DNA (other nucleic acid), 29 bp; single-stranded, linear
  description: /desc = “primer”
cggtctcccatgtgtggaattgttggcgc

SEQ ID NO: 2 - DNA (other nucleic acid), 34 bp; single-stranded, linear
  description: /desc = “primer”
ctctagagcgttgatattcagtcaattacaaaca

SEQ ID NO: 3 - DNA (other nucleic acid), 20 bp; single-stranded, linear
  description: /desc = “primer”
atggatgagcagacgatggt

SEQ ID NO: 4 - DNA (other nucleic acid), 19 bp; single-stranded, linear
  description: /desc = “primer”
cctcgaggtcgacggtatc